(19) United States
(12) Patent Application Publication
(10) Pub. No.: US 2026/0026775 A1
(43) Pub. Date: Jan. 29, 2026

(54) BLOOD PRESSURE DETECTION USING VESSEL DISTENSION AND PATIENT SIZE

(71) Applicant: Stryker Corporation, Portage, MI (US)

(72) Inventor: Clinton T. Siedenburg, Everett, WA (US)

(73) Assignee: Stryker Corporation, Portage, MI (US)

(21) Appl. No.: 19/281,091

(22) Filed: Jul. 25, 2025

Related U.S. Application Data
(60) Provisional application No. 63/676,032, filed on Jul. 26, 2024.

(51) Int. Cl. A61B 8/04; A61B 8/00; A61B 8/06; A61B 8/08

(52) U.S. Cl. CPC A61B 8/04; A61B 8/06; A61B 8/0891; A61B 8/488; A61B 8/5223

(57) ABSTRACT

An example method includes outputting, toward a blood vessel of a subject during a cardiac cycle of the subject, an ultrasound signal; detecting, from the blood vessel of the subject during the cardiac cycle of the subject, a reflection of the ultrasound signal; and determining a normalized distension of the blood vessel of the subject during the cardiac cycle by analyzing the reflection of the ultrasound signal. The example method further includes determining a blood pressure of the subject by adding a mean arterial pressure of the subject to a product of the normalized distension of the blood vessel and a pulse pressure of the subject. In addition, the example method includes outputting an indication of the blood pressure.

### CROSS-REFERENCE TO RELATED APPLICATION

This application claims priority to U.S. Provisional App. No. 63/676,032, which was filed on Jul. 26, 2024 and is incorporated by reference herein in its entirety.

### BACKGROUND

Blood pressure is an important vital sign that is indicative of a patient's health. Clinically, blood pressure often refers to both a systolic pressure, which refers to the maximum pressure measured during the cardiac cycle (e.g., one heartbeat), as well as a diastolic pressure, which refers to a minimum pressure of the cardiac cycle. Other clinically relevant blood pressure metrics include mean arterial pressure (MAP) and pulse pressure. Both MAP and pulse pressure are related to systolic and diastolic blood pressure. Blood pressure can be invasively measured using a catheter inserted into the patient's circulatory system. It can also be measured noninvasively by squeezing an extremity of the patient using an inflatable blood pressure cuff, and detecting the resultant pressure within the blood pressure cuff.

Existing techniques for blood pressure detection have a number of drawbacks. For example, invasive blood pressure measurements are less practical and more dangerous in out-of-hospital environments. Invasive blood pressure measurements also carry inherent risks for patients, even in controlled clinical environments, such as hospitals. Further, cuff-based blood pressure measurements are prone to error, particularly when the patient is in motion. In addition, cuff-based blood pressure methods require the physical restriction of the patient's extremity, which can be uncomfortable to the patient or even impossible if the patient's extremity is inaccessible. Further, cuff-based blood pressure methods are unable to safely detect the blood pressure in blood vessels located in parts of the patient's body that cannot be safely restricted, such as the neck. In addition, cuff-based blood pressure methods cannot safely detect the blood pressure of fetal blood vessels, or blood vessels within the umbilical cord.

Therefore, practical alternatives to existing blood pressure measurement techniques are needed.

### BRIEF DESCRIPTION OF THE DRAWINGS

FIG. 1 illustrates an example environment for determining blood pressure parameters of a subject using ultrasound.

FIG. 2 illustrates an example cross-section that intersects a blood vessel throughout a cardiac cycle.

FIG. 3 illustrates an example pressure waveform that can be generated using various techniques described herein.

FIG. 4 illustrates an example flow parameter waveform that can be generated using various techniques described herein.

FIG. 5 illustrates an example of a normalized width metric waveform that can be generated using various techniques described herein.

FIG. 6 illustrates an example process for noninvasively identifying the blood pressure of a subject using ultrasound.

FIG. 7 illustrates another example process for noninvasively determining a blood pressure parameter of a subject using ultrasound.

FIG. 8 illustrates an example of an external defibrillator configured to perform various functions described herein.

FIG. 9 illustrates a chest compression device configured to perform various functions described herein.

### DETAILED DESCRIPTION

Various implementations described herein relate to improved techniques for noninvasively detecting the blood pressure of a subject using ultrasound. In some cases, the Water Hammer equation, or formulas derived from the Water Hammer equation, can be used to identify a fluid pressure within a blood vessel based on instantaneous blood velocity, pulse wave velocity, and the density of the fluid. According to some examples, the Water Hammer equation can be adapted for determining blood pressure using ultrasound-detected parameters. For instance, ultrasound can be utilized to detect blood vessel wall movement due to a pulse traveling down the blood vessel at multiple locations along the blood vessel, which can be used to detect the velocity of the pulse (e.g., the pulse wave velocity). Moreover, ultrasound can be utilized to detect the velocity of blood in the blood vessel using a Doppler based technique. The density of blood, in some cases, can be estimated based on population-based trends. Accordingly, it may be theoretically possible to utilize ultrasound to estimate the blood pressure of a subject using the Water Hammer equation. However, in some examples, a discrepancy can exist between a Water Hammer-based estimation of blood pressure and the true blood pressure of a subject.

In various implementations of the present disclosure, an ultrasound-based blood pressure monitor is utilized to more accurately detect the blood pressure in a blood vessel of a subject by monitoring a cross-sectional shape of the blood vessel, as well as the blood flow within the vessel, over the course of a cardiac cycle. During the cardiac cycle, which includes systole and diastole, various geometric parameters of the blood vessel, as well as the velocity of the blood within the vessel, change according to the instantaneous blood pressure within the blood vessel. For instance, the cross-section of the blood vessel can be imaged using ultrasound. Due to the relatively high sampling rate of ultrasonic imaging, the cross-section can be imaged multiple times during the cardiac cycle. Also, the blood velocity can be imaged using ultrasound at a high sampling rate. Various types of blood pressure parameters can be detected by analyzing the images obtained during the cardiac cycle. In some implementations, various width metrics of the blood vessel are determined by analyzing the images. For example, techniques described herein can be used to detect the mean arterial pressure, the pulse pressure, and the systolic and diastolic blood pressures of the subject based on the images of the blood vessel, as well as blood flow measurements, throughout the cardiac cycle. Various systems, devices, and methods described herein can accurately, rapidly, and noninvasively detect one or more blood pressure metrics of a subject without constricting a limb of the subject. In some cases, techniques described herein enable accurate detection of the blood pressure within a blood vessel that is located outside of a limb that cannot be restricted using a blood pressure cuff.

Some techniques described herein can be utilized to enhance the accuracy of an ultrasound-based blood pressure metric measurement by considering the size of the subject. For instance, the size of the subject is indicative of a vascular load of the subject, which can impact a relationship between a calculated blood pressure metric (e.g., detected using ultrasound-based techniques) and a true blood pressure metric. Vascular load can refer to the aggregate resistance to blood flow prior to reaching the venous system, which can be dependent on characteristics of the network of arterioles and capillaries. In some cases, a device detects the size of the subject, for example, excess weight, excess body mass index, body fat percentage, or obesity. In some examples, a rescuer inputs an estimate of the size of the subject. A blood pressure metric, for instance, may be calculated based at least in part on the size of the subject.

Implementations of the present disclosure will now be described with reference to the accompanying figures.

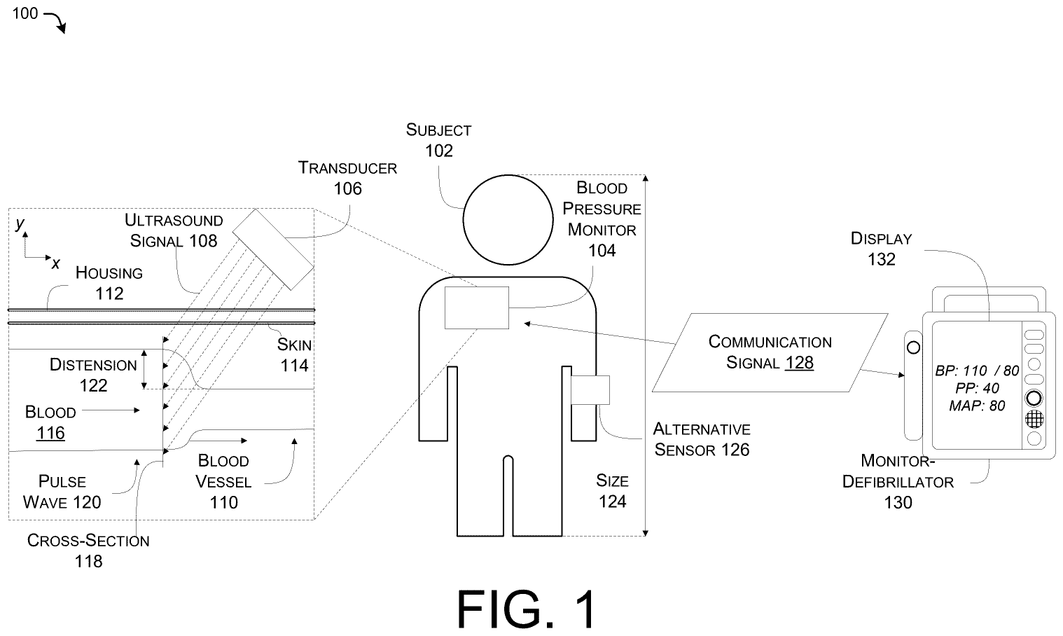

FIG. 1 illustrates an example environment 100 for determining blood pressure parameters of a subject 102 using ultrasound. In some examples, the subject 102 is a patient in a clinical environment. In some cases, the subject 102 is a patient in a non-clinical environment, such as a rescue scene. For instance, the subject 102 may have experienced a medical emergency (e.g., cardiac arrest, loss-of-consciousness, acute injury, etc.) in an out-of-hospital environment. In some cases, the subject 102 is in a home environment. In various examples, it may be beneficial to assess the blood pressure of the subject 102.

A blood pressure monitor 104 is configured to detect one or more blood pressure parameters of the subject 102. The blood pressure monitor 104, for instance, is disposed on the skin of the subject 102. In some examples, the blood pressure monitor 104 is affixed to the skin of the subject 102 via a biocompatible adhesive. In some cases, the blood pressure monitor 104 is strapped to the body of the subject via one or more straps.

Conventionally, the blood pressure of the subject 102 can be detected using a blood pressure cuff. However, the blood pressure cuff has multiple drawbacks and limitations. First, the operation of the blood pressure cuff includes inflating or otherwise tightening around a limb (e.g., an arm) of the subject 102. In various cases, the tightening of the blood pressure cuff can produce discomfort and anxiety in the subject 102. In emergency situations in which the limb of the subject 102 is acutely injured or inaccessible, it may be dangerous or impossible to cause the blood pressure cuff to tighten around the limb of the subject 102. Second, measurements of the blood pressure cuff are relatively inaccurate. Third, in some examples, the blood pressure cuff has a blood pressure sampling period on the order of minutes. Accordingly, the blood pressure cuff may be unable to detect sudden changes in the blood pressure of the subject 102 with respect to time.

In various implementations of the present disclosure, the blood pressure monitor 104 assesses the condition of the subject non-invasively using ultrasound. Unlike the blood pressure cuff, the blood pressure monitor 104 is capable of accurately detecting one or more blood pressure metrics of the subject 102 during each cardiac cycle of the subject 102. Furthermore, the blood pressure monitor 104 can measure the blood pressure metric(s) without tightening around the limb of the subject 102. For instance, in some cases, the blood pressure monitor 104 can be applied to the neck of the subject 102 in order to detect blood pressure metric(s) of one or more blood vessels in the neck of the subject 102.

The blood pressure monitor 104 includes a transducer 106 configured to emit an ultrasound signal 108 toward a blood vessel 110 of the subject 102. As used herein, the term “ultrasound,” and its equivalents, can refer to mechanical waves (e.g., in the form of pressure waves) having a frequency in a range of 20 kilohertz (kHz) to 200 megahertz (MHz). Ultrasound, for example, is sound in a frequency that is greater than an upper detection limit of a human ear. In various instances, the transducer 106 includes one or more piezoelectric crystals (including, e.g., lead zirconate titanate (PZT), LiNbO₃ (LN), lead magnesium niobate-lead titanate (PMN-PT), or lead indium niobate-lead magnesium niobate-lead titanate (PIN-PMN-PT)), or similar piezoelectric crystals. When an electrical voltage pulse is applied across the piezoelectric crystal(s), the piezoelectric crystal(s) vibrate at a frequency that produces ultrasound. In some examples, the transducer 106 includes one or more micro-electromechanical system (MEMS) devices. In some instances, the transducer 106 includes one or more capacitive micromachined ultrasonic transducers (CMUTs) and/or piezoelectric micromachined ultrasonic transducers (PMUT). Any electrical to mechanical conversion system or material that operates at the ultrasound frequency range can be included in the transducer 106.

In some cases, the transducer 106 includes a transducer element (e.g., a piezoelectric crystal, MEMS device, or another type of electrical-to-mechanical conversion device), a first electrode disposed on one side of the transducer element, and a second electrode disposed on another side of the transducer element. In various implementations, the transducer 106 is configured to produce the ultrasound signal 108 by inducing a current through or a voltage between the first and second electrodes. In some cases, the transducer 106 is configured to detect a reflection of the ultrasound signal 108 by detecting a current through or voltage between the first and second electrodes that is induced when the reflection of the ultrasound signal 108 is received by the transducer element.

In some cases, the transducer 106 is encased in a housing 112, which may be watertight. According to various cases, the ultrasound signal 108 is transmitted through the housing 112. The housing 112, for instance, includes a polymer that is transmissible to ultrasound. The transducer 106, in some examples, further includes a matching layer that is disposed between the transducer element and a surface of the housing 112 from which an incident beam of the ultrasound signal 108 is emitted. The matching layer includes a material having an acoustic impedance that is between the acoustic impedance of the transducer element and an acoustic lens (e.g., if one exists or between the transducer 106 and the skin 114 of the subject 102). In some implementations, a gel layer is disposed between the housing of the ultrasound transducer and the skin 114 that further matches the impedance between the ultrasound transducer and the body, thereby preventing the ultrasound signal 108 from being reflected by an interface containing air between the skin 114 and the transducer 106. In some cases, an adhesive adhering the blood pressure monitor 104 to the skin 114 of the subject 102 serves as the gel layer.

The blood vessel 110 is located in the body of the subject 102 and transports blood 116 from the heart of the subject 102 or to the heart of the subject 102. In some implementations, the blood vessel 110 is an artery. In various cases, the artery carries oxygenated blood 116 from the heart of the subject 102. Examples of the artery include a carotid artery, a subclavian artery, a coronary artery, a brachial artery, an iliac artery, a radial artery, a femoral artery, or a pulmonary artery. In some cases, the pulmonary artery carries deoxygenated blood 116. In some examples, the blood vessel 110 is a vein that carries deoxygenated blood 116 toward the right chamber of the heart and the pulmonary artery of the subject 102. Examples of the vein include a jugular vein, an iliac vein, a subclavian vein, a cephalic vein, a brachial vein, a basilic vein, a hepatic vein, a radial vein, an ulnar vein, a digital vein, a brachiocephalic vein, a femoral vein, a saphenous vein, a venous arch, or a tibial vein.

In some examples, the ultrasound signal 108 is transmitted at an angle with respect to the housing 112 and/or the skin 114 of the subject 102. The angle, for instance, is not a 90 degree angle. In some examples, at least a component of the ultrasound signal 108 is parallel to the housing 112 and/or the skin 114 of the subject 102. In some cases, at least a portion of the ultrasound signal 108 is transmitted at a 90 degree angle with respect to the housing 112 and/or the skin 114 of the subject 102. For example, the ultrasound signal 108 may include beams transmitted at different directions, or a lobe that is output at a range of directions, with respect to the housing 112 and/or the skin 114.

In various implementations, the ultrasound signal 108 is transmitted at an angle with respect to the flow of the blood 116 in the blood vessel 110. The angle, for example, is not a 90 degree angle. In some implementations, at least a component of the ultrasound signal 108 is parallel to the flow of the blood 116 in the blood vessel 110. In some cases, at least a portion of the ultrasound signal 108 is transmitted at a 90 degree angle with respect to the blood vessel 110.

In various implementations, the ultrasound signal 108 is reflected and/or scattered by the blood 116 in the blood vessel 110, as well as by other tissues along a cross-section 118 that intersects the blood vessel 110. In various cases, the transducer 106 is configured to detect a reflection of the ultrasound signal 108 from the blood and/or tissues along the cross-section 118. The blood pressure monitor 104, for instance, is configured to detect characteristics of the blood vessel 110 based on the reflection of the ultrasound signal 108 detected by the transducer 106.

In various implementations, the blood pressure monitor 104 is configured to detect a parameter indicative of the flow of the blood 116 through the blood vessel 110 based on the reflection of the ultrasound signal 108. According to various cases, the blood pressure monitor 104 is configured to detect an instantaneous velocity of the blood 116 in the blood vessel 110 by detecting a Doppler shift between the ultrasound signal 108 and the reflection of the ultrasound signal 108. In examples in which the transducer 106 operates in a continuous wave mode, the Doppler shift may be based on a difference between the frequency of the ultrasound signal 108 and the frequency of the reflection of the ultrasound signal 108. In some examples in which the transducer 106 operates in a pulsed wave mode, the Doppler shift may be based on a difference between the phase of pulses of the ultrasound signal 108 and the phase of the reflections of the pulses of the ultrasound signal 108. In various implementations, the blood pressure monitor 104 is configured to detect the velocity of the blood 116 at one or more locations along the cross-section based on the Doppler shift.

In some examples, the blood pressure monitor 104 is configured to detect motion associated with the blood vessel 110 and/or the blood 116 using speckle tracking. For instance, the blood pressure monitor 104 may be configured to generate 2D or 3D images of the blood vessel over time using the ultrasound signal 108. In various implementations, the blood pressure monitor 104 may identify one or more speckles that are depicted in each of the 2D or 3D images. A speckle, for instance, may be a shape in a 2D or 3D image of a tissue that is indicative of an arbitrary structure in the tissue. For instance, the blood pressure monitor 104 may be configured to detect one or more speckles in the depictions of the blood 116 in the blood vessel 110 in the 2D or 3D images (e.g., representing cross-sections of the blood vessel 110 captured along a longitudinal axis of the blood vessel 110). The blood pressure monitor 104, in various examples, is configured to track motion of the blood 116 in the blood vessel 110 by comparing the pixel locations of the one or more speckles across the 2D or 3D images. For instance, the blood pressure monitor 104 may be configured to detect the velocity of the blood 116 by tracking the one or more speckles in the 2D or 3D images captured over time.

As used herein, the terms “blood flow,” “blood flow parameters,” and their equivalents, may refer to one or more physiological parameters indicative of a movement of blood through a blood vessel. For example, the term “blood velocity” may refer a change in position with respect to time (i.e., distance per time, such as meters per second) of one or more blood cells moving in a blood vessel. The term “blood speed” may refer to a magnitude of a velocity (i.e., distance per time) of one or more blood cells moving in a blood vessel. The term “velocity profile,” for example, may refer to a velocity of blood in a blood vessel with respect to a location within the blood vessel, such as a velocity of blood in a blood vessel with respect to a location along a cross-section of the blood vessel. The term “flow rate,” and its equivalents, may refer to a volume or mass of blood that passes a boundary (e.g., a cross-section of a blood vessel) with respect to time. The terms “net flow,” “net flow volume,” and their equivalents, may refer to a volume or mass of blood that passes a boundary during a discrete time interval, such as during a cardiac cycle. A net flow volume can be calculated by integrating a flow rate over the time interval. Unless otherwise specified explicitly or by context, the term “blood flow” may refer to blood velocity, blood speed, velocity profile, flow rate, net flow, or any other flow-related parameter described herein.

The blood pressure monitor 104, in various cases, is configured to detect a width metric of the blood vessel 110 based on the reflection of the ultrasound signal 108. According to some cases, the width metric is calculated based on a reflection of a portion of the ultrasound signal 108 that is substantially perpendicular to the blood vessel 110. The term “width metric,” and its equivalents, may refer to any parameter that is indicative of a cross-sectional size of a blood vessel. Examples of the width metric include, for instance, a width, a radius, a diameter, a circumference, a perimeter length, a cross-sectional area, a volume of a segment of the blood vessel, or any combination thereof. In some cases, the width metric is represented by a waveform over time. According to some implementations, the ultrasound signal 108 is reflected from a wall of the blood vessel 110. In some examples, the ultrasound signal 108 is reflected from an interior wall of the blood vessel 110 and/or an exterior wall of the blood vessel 110. By detecting the reflection of the ultrasound signal 108 from the wall of the blood vessel 110, the blood pressure monitor 104 may detect the width metric of the blood vessel 110 along the cross-section 118. In some examples, the blood pressure monitor 104 may infer the width metric of the blood vessel 110 by analyzing the reflection of the ultrasound signal 108 from a tissue adjacent to the wall of the blood vessel 110.

In various cases, the blood pressure monitor 104 is configured to determine multiple cross-sectional images (e.g., 1D, 2D, and/or 3D images of the cross-section 118) of the blood vessel 110 over time (e.g., multiple times during the cardiac cycle) based on the reflection of the ultrasound signal 108. In various cases, the cross-sectional images include digital images that are indicative of the cross-section 118 of the blood vessel 110 at different sampling times. Further, the blood pressure monitor 104 may determine the width metric and/or the change in the width metric of the blood vessel 110 by analyzing the cross-sectional images. The cross-sectional images, for example, may be associated with the phase and frequency of one or more portions of the ultrasounds signal 108 utilized to generate various portions of the cross-sectional images. The phase and/or frequency information can be further utilized to determine the width metric and/or the change in the width metric. Various techniques can be utilized to derive the width metric and/or the change in width metric based on the cross-sectional images. In some instances, the blood pressure monitor 104 may identify the portions of the cross-sectional images that are indicative of the wall of the blood vessel 110 using edge detection. For example, the blood pressure monitor 104 may detect pixels in the images indicative of the wall of the blood vessel 110 using at least one of Canny edge detection, edge thinning, gradient detection, Kovalevsky edge detection, Marr-Hildreth edge detection, a phase stretch transform, or any combination thereof. Subsequently, the blood pressure monitor 104 may estimate the width metric by determining one or more distances between the pixels depicting the wall of the blood vessel 110 in the images. For instance, a diameter of the blood vessel 110 can be derived by determining a maximum distance between the pixels depicting the wall of the blood vessel 110. The width metric, in some cases, can be in terms of pixel lengths and/or in terms of an absolute length (e.g., in millimeters (mm) or centimeters (cm)). The absolute length can be derived based by calibrating the blood pressure monitor 104 before the cross-sectional images are obtained.

In various implementations, the blood flow and width metric of the blood vessel 110 periodically changes based on a cardiac cycle of the subject 102. During systole, the heart muscle contracts and propels the blood 116 through vasculature of the subject 102, including the blood vessel 110. During diastole, the heart muscle relaxes and fills with the blood 116. The cardiac cycle of the subject 102 is repeated for each heartbeat. The activation of the heart muscle during the cardiac cycle produces an electrical signal that can be detected via an electrocardiogram (ECG). Assuming that the subject 102 does not have an arrhythmia or other medical condition, the cardiac cycle produces cyclical changes in the blood flow, blood pressure, and geometry (e.g., width) of the blood vessel 110 with respect to time. Notably, the resultant changes in blood flow, blood pressure, and blood vessel 110 geometry may be slightly delayed compared to the actions of the heart of the subject 102, depending on the distance along the vasculature of the subject 102 between the heart and the blood vessel 110.

The blood flow through the blood vessel 110 exhibits a first peak during systole and a second peak during diastole. In various cases, the blood flow through the blood vessel 110 has a greater first peak during systole than the second peak during diastole.

The width metric of the blood vessel 110 varies based on a pulse wave 120 that is transmitted along the blood vessel 110. Although a single pulse wave 120 is depicted in FIG. 1, it should be appreciated that the systole produces a first pulse wave and diastole produces a second pulse wave, in some cases. In some implementations, the pulse wave 120 can be induced by another mechanical stimulus, such as an ultrasound pulse or physical push. The pulse wave 120 travels along the blood vessel 110 at a velocity referred to as the pulse wave velocity (PWV). As the pulse wave 120 crosses the cross-section 118, the width metric of the blood vessel 110 along the cross-section 118 temporarily increases.

In various cases, the pulse wave 120 causes a measurable distension 122 of the blood vessel 110 that can be detected based on the reflection of the ultrasound signal 108. As a result of the distension 122, the width metric of the blood vessel 110 changes over time. In some examples, the blood pressure monitor 104 is configured to perform phase measurements of a waveform representing the distension of the blood vessel 110 over time. For instance, the blood pressure monitor 104 may detect a phase difference (as a function of time) between opposing sides of the wall of the blood vessel 110 and/or immediately adjacent tissues. The phase difference may be normalized to unit amplitude and mean of zero to estimate the shape of the blood pressure waveform.

According to various implementations, the blood pressure monitor 104 is configured to sample the blood flow through the blood vessel 110 and the width metric of the blood vessel 110 multiple times during the cardiac cycle of the subject 102. In some cases, the blood pressure monitor 104 samples the blood flow and the width metric at one or more sampling rates, such as 5 Hz, 10 Hz, 50 Hz, 100 Hz, 200 Hz, 1 kHz, 2 kHz, 3 kHz, 4 kHz, 5 kHz, 10 kHz, or the like. In some cases, the blood pressure monitor 104 samples the blood flow and the width metric during multiple cardiac cycles of the subject 102. According to various cases, the blood pressure monitor 104 is configured to generate a waveform of the blood flow through the blood vessel 110 over time for each cardiac cycle and/or a waveform of the width metric of the blood vessel 110 over time for each cardiac cycle. The blood pressure monitor 104, in some cases, samples the width metric of the blood vessel 110 by tracking different pixels (indicating intensity and/or phase of the portions of the ultrasound signal 108 utilized to generate the pixels) representative of the wall of the blood vessel 110, such that the blood pressure monitor 104 may generate multiple waveforms of the width metric during the same cardiac cycles. In some implementations, the blood pressure monitor 104 generates a combination of waveforms representative of the same parameter over time and captured during the same and/or different cardiac cycles.

According to some implementations, the blood pressure monitor 104 is configured to detect the PWV based on the reflection of the ultrasound signal 108. In some cases, the blood pressure monitor 104 is configured to detect a time at which the pulse wave 120 crosses the cross-section 118 by detecting movement in the wall of the blood vessel 110. For instance, the blood pressure monitor 104 is configured to detect when a Doppler shift of the reflection of the ultrasound signal 108 from the wall of the blood vessel 110 exceeds a threshold. In various cases, the blood pressure monitor 104 also identifies a time at which the pulse wave 120 crosses another cross-section that intersects the blood vessel 110. For instance, the blood pressure monitor 104 may detect a time at which a reflection of an ultrasound signal produced by another transducer indicates that the pulse wave 120 has passed the other cross-section. The blood pressure monitor 104 may determine the PWV by dividing the length between the cross-section 118 and the other cross-section by the difference between the times. According to some cases, the blood pressure monitor 104 derives the PWV based at least in part on a time at which the heart of the subject 102 produces the pulse wave 120 (e.g., a time at which the heart is in systole). For example, the blood pressure monitor 104 may include an ECG sensor configured to detect a QRS complex of the subject 102 and/or a microphone configured to detect a heart sound of the subject 102 that is indicative of a portion of the cardiac cycle that has produced the pulse wave 120. The blood pressure monitor 104, in some cases, identifies the PWV by dividing the distance between the heart of the subject 102 and the cross-section 118 along the vasculature of the subject 102 by a difference between the time of the QRS complex and/or the heart sound and the time that the pulse wave 120 has crossed the cross-section 118. In some implementations, the blood pressure monitor 104 is configured to determine a derivative of a pressure waveform crossing a single cross-section of the blood vessel 110 in order to determine the PWV.

The blood pressure monitor 104, for instance, is configured to generate the one or more blood pressure metrics based, at least in part, on the blood flow through the blood vessel 110, the width metric of the blood vessel 110, the PWV, or any combination thereof. The term “blood pressure metric” may refer to any parameter that is representative of a type of blood pressure of the subject 102.

In various examples, the blood pressure monitor 104 is configured to detect a pulse pressure of the subject 102. The term “pulse pressure” may refer to a difference between a systolic blood pressure and a diastolic blood pressure. For instance, the pulse pressure of the subject 102 is dependent on the maximum change of pressure in the blood vessel 110 that the heart of the subject 102 produces each time it transitions from the end of diastole to the peak of systole. In various cases, the pulse pressure can be calculated as a function of a change in the blood flow through the cross-section 118 over the course of the cardiac cycle as well as the PWV of the pulse wave 120. According to some examples, the pulse pressure is dependent on the change of a velocity of the blood 116 (e.g., through the centroid of the internal area of the blood vessel 110 that has intersected the cross-section 118) between the systolic phase and the diastolic phase of the cardiac cycle. In some implementations, the pulse pressure is estimated by identifying an entry of a look-up table (or one or more empirically generated equations) corresponding to the change in the blood flow as well as the PWV. The pulse pressure, for instance, increases as the change in the blood flow increases. Further, the pulse pressure, in some cases, increases as the PWV increases.

In various implementations, the blood pressure monitor 104 is configured to detect a mean arterial pressure (MAP) of the subject 102. In examples in which the blood vessel 110 is an artery, the “MAP” may refer to an average blood pressure in the blood vessel 110 during a single cardiac cycle. According to various cases, the blood pressure monitor 104 calculates the MAP as a function of the mean of the blood flow through the blood vessel 110 during the cardiac cycle. In some cases, the function to calculate the MAP is further based on the width metric of the blood vessel 110. In some examples, the function is based on the minimum or maximum or average (e.g., mean, median, mode, etc.) of the width metric (e.g., diameter) of the blood vessel 110 throughout the cardiac cycle. According to some cases, the MAP increases as the mean of the blood flow through the blood vessel 110 increases. The MAP may decrease as the width metric of the blood vessel 110 increases. According to some examples, the blood pressure monitor 104 estimates the MAP by identifying an entry of a look-up table (or one or more empirically generated equations) corresponding to the mean blood flow and/or the width metric of the blood vessel 110.

In particular cases, the blood pressure monitor 104 is configured to detect a blood pressure in the blood vessel 110. The blood pressure may vary over time during the cardiac cycle of the subject 102. For instance, the blood pressure may be represented as a waveform. According to various implementations, the blood pressure waveform has a similar shape to a waveform representing the width metric (e.g., distension) of the blood vessel 110 over the course of the cardiac cycle. In some cases, the blood pressure monitor 104 identifies the blood pressure waveform based, at least in part, on normalizing the width metric waveform. In some cases, the blood pressure monitor 104 subtracts a minimum of the width metric waveform from each sample of the width metric waveform, such that the resultant waveform has a minimum of 0. In some examples, the blood pressure monitor 104 divides each sample of the resultant waveform by a maximum change in the width metric of the course of the cardiac cycle. An intermediary waveform may be generated by dividing the waveform by the maximum change. In some cases, the blood pressure monitor 104 further subtracts the mean of the width metric over the course of the cardiac cycle from the intermediary waveform. As a result, the blood pressure monitor 104 may generate a waveform representing the normalized width metric of the blood vessel 110 over the course of the cardiac cycle.

According to various cases, the blood pressure monitor 104 generates a waveform representing the blood pressure in the blood vessel 110 based on the waveform representing the normalized width metric of the blood vessel 110. In various examples, the blood pressure monitor 104 generates a product of the pulse pressure and the waveform representing the normalized width metric of the blood vessel 110. The blood pressure waveform, for instance, can be generated by adding the MAP to each sample of the product. PWV, diastolic to systolic rise in blood flow (velocity), mean vessel width, mean blood flow (e.g., velocity), and other computed parameters described herein can be estimated on the same cardiac cycle to generate a blood pressure parameter. In some cases, generated blood pressure parameters can be averaged over multiple cardiac cycles. Alternatively, each of the contributing parameters could be averaged (e.g., mean, median, or mode) independently over multiple cardiac cycles before the blood pressure parameter is computed, and can be applied to the distension waveform. Moreover, the distension waveform could also be generated from some average or filtering over time.

In some cases, the blood pressure monitor 104 is configured to detect a systolic blood pressure, which is typically equal to a peak blood pressure during the systolic phase of the cardiac cycle. In various implementations, the blood pressure monitor 104 detects the systolic blood pressure by identifying a maximum of the blood pressure waveform over the cardiac cycle In some examples, the blood pressure monitor 104 is configured to detect a diastolic blood pressure, which is typically equal to a minimum blood pressure during the end of the diastolic phase of the cardiac cycle. In some cases, the blood pressure monitor 104 detects the diastolic blood pressure by identifying a minimum of the blood pressure waveform over the cardiac cycle.

In various cases, the blood pressure metric(s) are dependent on the size 124 of the subject 102. In particular implementations, the blood pressure metric(s) are dependent on whether the size 124 of the subject 102 is dependent on excess body fat, musculature, or other soft tissues. Physiologically, the size 124 of the subject 102 is related to the load on the heart of the subject 102 that is provided by the vasculature of the subject 102. Various blood pressure metrics described herein (e.g., pulse pressure, MAP, etc.) are related to the vasculature load of the subject 102. In various examples, the blood pressure monitor 104 identifies a metric indicative of the size 124 of the subject 102. In some cases, the size 124 is indicated by a height, a weight, a body mass index (BMI), a body fat percentage, a height-weight ratio (e.g., a height of the subject 102 divided by a weight or mass of the subject 102), a clothing size (e.g., a shirt size, a pants size, a dress size, etc.), a circumference (e.g., of the neck of the subject 102, of the waist of the subject 102, of the wrist of the subject 102, etc.), a volume of at least a portion of the subject 102, or some other indication. In some cases, the size 124 indicates whether or not the subject 102 is pregnant. In some examples, the blood pressure monitor 104 receives an input signal (e.g., from a user, such as a rescuer) that indicates the size 124 of the subject 102. For instance, the user may measure, estimate, or otherwise identify the size 124 of the subject 102. In some implementations, the blood pressure monitor 104 is communicatively coupled to an electronic medical record (EMR) device, which may communicate the size 124 of the subject 102 as indicated in an EMR of the subject 102.

In various implementations of the present disclosure, the blood pressure monitor 104 identifies one or more blood pressure metrics of the subject 102 based, at least in part, on the size 124 of the subject 102. For instance, the function utilized by the blood pressure monitor 104 to calculate pulse pressure or MAP may further depend on the metric indicative of the size 124 of the subject 102. In some examples, the look-up table (or empirically derived equation) utilized by the blood pressure monitor 104 to identify the pulse pressure or MAP may further depend on the size 124 of the subject 102.

According to some examples, the blood pressure monitor 104 selectively detects the characteristic(s) of the blood vessel when the blood pressure monitor 104 is moving less than a threshold amount. For instance, the blood pressure monitor 104 may include a motion sensor (e.g., an accelerometer, gyroscope, etc.) that detects a metric indicative of motion of the blood pressure monitor 104 relative to the blood vessel 110. In some cases, the relative motion can result in artifact in one or more detected signals of the transducer 106. For example, the relative motion could distort the Doppler shift detected by the blood pressure monitor 104. It could also add artifacts to the Doppler measurement of blood flow, Furthermore, it may also corrupt the measurement of the cross section. It may therefore be beneficial to avoid reliance on blood pressure metrics derived based on signals detected by the transducer 106 when the transducer 106 is moving relative to the blood vessel 110 and/or the tissue adjacent.

In some cases, the blood pressure monitor 104 is communicatively coupled with an alternative sensor 126 configured to detect blood pressure using a different methodology than the blood pressure monitor 104. For instance, the alternative sensor 126 may include a catheter sensor configured to detect the blood pressure invasively, a blood pressure cuff, an oscillometric blood pressure sensor, or any combination thereof. In some cases, the blood pressure monitor 104 corrects the blood pressure measurement it performs based on the blood pressure identified by the alternative sensor 126. In some cases, the blood pressure monitor 104 ignores the blood pressure metric(s) generated based on the ultrasound signal 108 during conditions in which it infers that the blood pressure monitor 104 is detecting blood pressure inaccurately. For instance, the blood pressure monitor 104 may at least temporarily rely on the blood pressure detected by the alternative sensor 126 when the motion of the blood pressure monitor 104 is greater than a threshold, there is greater than a threshold amount of artifact in the blood pressure waveform, or the like. In some cases, the blood pressure monitor 104 combines the blood pressure measurements generated based on the ultrasound signal 108 and the alternative sensor 126 to provide increased robustness to the blood pressure estimation.

In some conditions, an ultrasound-based blood pressure measurement by the blood pressure monitor 104 has a greater accuracy than a cuff-based blood pressure measurement. For instance, vehicle motion, patient motion, and vibration can result in erroneous readings by an oscillatory blood pressure device. Thus, in cases in which the acceleration of the blood pressure monitor 104 and/or the alternative sensor 126 is greater than a threshold, the monitor-defibrillator 130 may rely exclusively on the measurement performed by the blood pressure monitor 104 (i.e., an estimated blood pressure detected by the blood pressure monitor 104, rather than an estimated blood pressure detected by the alternative sensor 126). In some cases, anxiety induced by the constriction of the limb of the subject 102 by the subject 102 causes errors and inaccuracies in blood pressure measurements by the cuff-based alternative sensor 126. Thus, if the subject 102 is prone to anxiety during a cuff-based blood pressure measurement, the measurement performed by the blood pressure monitor 104 may more reliably represent the condition of the subject 102. Various implementations described herein can be applied to not only emergency care environments, but also clinical environments, home monitoring, fitness monitoring, intensive care, and personal health monitoring.

According to some examples, the blood pressure monitor 104 outputs an indication of the characteristic(s) and/or the blood pressure metric(s). In some cases, the blood pressure monitor 104 includes a display configured to visually present the characteristic(s) and/or blood pressure metric(s). For instance, the blood pressure monitor 104 may display the waveform representative of the blood pressure in the blood vessel 110 over the course of the cardiac cycle. In some examples, the blood pressure monitor 104 includes a speaker configured to audibly report the characteristic(s) and/or blood pressure metric(s). In some implementations, the blood pressure monitor 104 outputs an alert upon determining that the characteristic(s) and/or the blood pressure metric(s) are below a first threshold or above a second threshold. The alert, for instance, may be visual or audible.

In some cases, the blood pressure monitor 104 transmits, to an external device, a communication signal 128 indicating the characteristic(s) and/or the blood pressure metric(s). In some cases, the communication signal 128 is transmitted over a wireless interface. For instance, the communication signal 128 may be a BLUETOOTH™ signal. The communication signal 128, for instance, encodes data indicative of the characteristic(s) and/or the blood pressure metric(s). In some cases, the communication signal 128 includes multiple transmissions that collectively report ongoing measurements identified by the blood pressure monitor 104. In various examples, the communication signal 128 indicates raw data (e.g., measurements detected without further processing), partially processed data (e.g., base banded data), processed data (e.g., blood pressure waveforms), or any combination thereof, detected by the blood pressure monitor 104.

In some examples, the blood pressure monitor 104 transmits the communication signal 128 to a monitor-defibrillator 130. For instance, the monitor-defibrillator 130 includes a display 132 configured to visually present the characteristic(s) and/or the blood pressure metric(s). In some examples, the display 132 is configured to visually present raw data and/or base banded data detected by the blood pressure monitor 104. In some examples, the monitor-defibrillator 130 calculates the blood pressure metric(s) based on the reported characteristic(s), in accordance with the techniques described herein.

In some implementations, the blood pressure monitor 104 is configured to determine one or more metrics described herein (e.g., width metrics, blood pressure metrics, any acoustically derived metric described herein, etc.) by taking an average (e.g., median, mean, etc.) of multiple measurements. In some cases, the blood pressure monitor 104 selectively outputs, or reports, a running average of multiple (e.g., five, ten, etc.) measurements acquired over time. By aggregating the measurements in the form of an average, the quality of the metrics output by the blood pressure monitor 104 may be enhanced.

In various cases, the blood pressure monitor 104 is configured to determine a quality metric associated with various metrics or measurements described herein. For example, the blood pressure monitor 104 is configured to determine a variance, Z-score, or other statistic indicative of the quality of a given metric or measurement. In some cases, the blood pressure monitor 104 refrains from outputting a metric that is associated with a quality metric that is below a predetermined threshold. In some examples, the blood pressure monitor 104 outputs an average of multiple measurements or calculations that are averaged in view of their respective quality metrics. For instance, the blood pressure monitor 104 may calculate five estimates of the pulse pressure, taken over five separate instances of the cardiac cycle. The blood pressure monitor 104 may determine a Z-score associated with each of the five estimates in view of each other. Further, the blood pressure monitor 104 may output an average of the estimates of pulse pressure, weighted in view of the respective Z-scores, such that estimates with a lower Z-score are more represented in the average than estimates with a higher Z-score. Various implementations of the present disclosure reduce the impact of noise on the accuracy of reported metrics.

In some implementations, the blood pressure monitor 104 further indicates a confidence (e.g., the quality metric) associated with any metric it reports. For example, the blood pressure monitor 104 may output an indication of the variance of blood pressure metric measurements used to generate an average blood pressure metric, along with the average blood pressure metric, in order to report a reliability of the average blood pressure metric as-reported. In some cases, the blood pressure monitor 104 displays the average blood pressure metric with a color associated with the variance, such that the average blood pressure metric may be displayed in red if it is based off of measurements with a high variance or the average blood pressure metric may be displayed in green if it is based off of measurements with a low variance. In some cases, the confidence associated with the reported metric is numerically reported.

Although FIG. 1 has been described such that the blood pressure monitor 104 utilizes ultrasound to calculate various metrics described herein, implementations of the present disclosure are not so limited. For instance, it should be understood that various applications of ultrasound described herein can be substituted with light-based techniques. In particular cases, the ultrasound signal 108 can be substituted with light. In various cases, the light is at least one of infrared, near-infrared, or visible light. For instance, the light may have a frequency in a range of 300 GHz-430 THz and a wavelength in a range of 700 nanometers (nm) to 1 millimeter (mm). For instance, the transducer 106 includes, or is substituted with, one or more light sources, such as light-emitting diodes (LEDs) or lasers. In some examples, the one or more mirrors are configured to split the light output by the light source(s), such as for an interferometric analysis. In some cases, transducer 106 includes, or is substituted with, one or more light sensors, such as at least one of a photodiode, a phototransistor, a photomultiplier tube, a charge-coupled device, a metal-semiconductor-metal photodetector, or a complementary metal oxide semiconductor photodetector.

In some examples, the blood pressure monitor 104 performs laser Doppler velocimetry in order to detect the blood velocity. In various cases, the blood pressure monitor 104 includes one or more interferometric sensors. For example, the blood pressure monitor 104 outputs light beams (e.g., coherent light beams) that are split (e.g., by one or more mirrors) prior to transmission through the skin 114. The blood pressure monitor 104 may detect the blood velocity in the blood vessel 110 by comparing a return of the first beam and a return of the second beam to the split beams generated from the first incident beam and the second incident beam. Techniques for interferometric detection of blood velocity can be found in, for example, R. D. Rader, C. M. Stevens and J. P. Meehan, “An Interferometric Blood Flow Measurement Technique—A Brief Analysis,” in IEEE Transactions on Biomedical Engineering, vol. BME-21, no. 4, pp. 293-297, July 1974; Nagahara, et al., Method. Invest. Ophthalmol. Vis. Sci. 2011; 52(1):87-92; and Robinson, et al., Sci Rep 13, 8803 (2023), each of which is incorporated by reference herein in its entirety. Similarly, the blood pressure monitor 104 may perform laser Doppler velocimetry in order to detect movement of the blood vessel 110 in order to identify PWV.

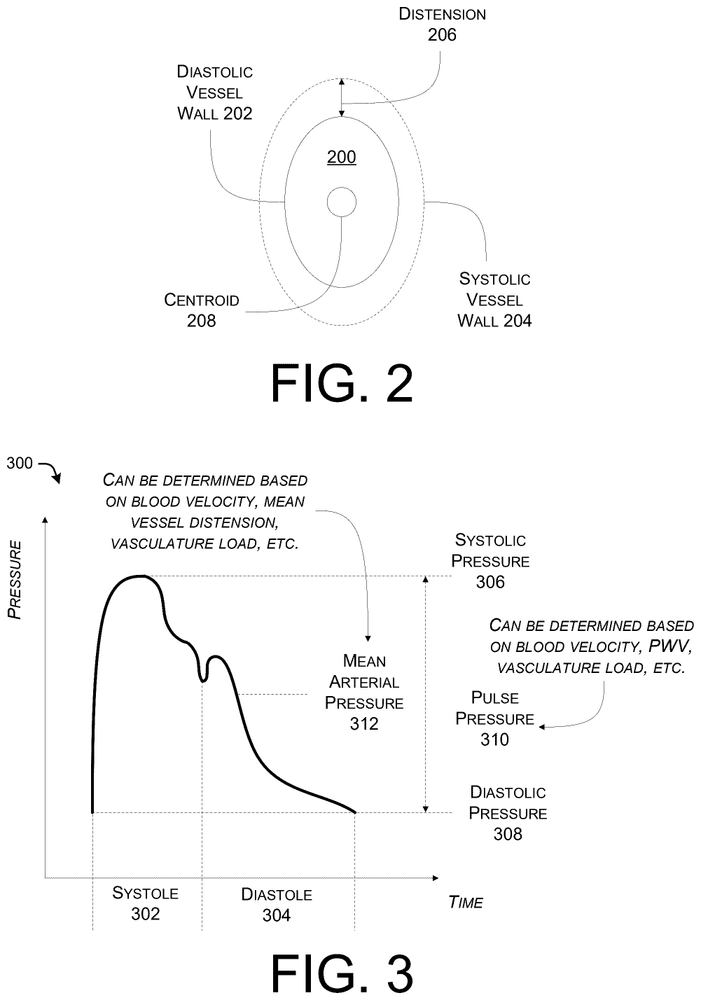

FIG. 2 illustrates an example cross-section that intersects a blood vessel 200 throughout a cardiac cycle. For instance, the blood vessel 200 illustrated in FIG. 2 may be the blood vessel 110. The cross-section, for example, may be the cross-section 118.

A width metric of the blood vessel changes throughout the cardiac cycle. For example, the blood vessel 200 may be bounded by a diastolic vessel wall 202 during a diastolic phase of the cardiac cycle, and may be bounded by a systolic vessel wall 204 during a systolic phase of the cardiac cycle. In various cases, a width metric (e.g., a width, radius, diameter, area, or the like) of the blood vessel 200 in the systolic phase is greater than the width metric of the blood vessel 200 in the diastolic phase. In various cases, distension 206 is defined as a difference between the width metric of the diastolic vessel wall 202 to the time-varying location of the vessel wall which will be a maximum at the systolic vessel wall 204.

Various blood pressure metrics described herein can be calculated based on the width metric of the diastolic vessel wall 202, the width metric of the systolic vessel wall 204, the distension 206, or any combination thereof. According to some examples, the blood vessel 110 is an artery and the MAP within the blood vessel 200 is dependent on the width metric of the diastolic vessel wall 202, the width metric of the systolic vessel wall 204, the distension 206, or any combination thereof. According to some cases, a waveform representing the blood pressure in the blood vessel 200 with respect to time (e.g., during the cardiac cycle) is generated based on a waveform representing the normalized distension 206 with respect to time.

In some cases, the blood pressure metrics can be calculated, based at least in part, on a flow parameter of blood in the blood vessel 200. In some cases, the blood pressure metrics are based on a velocity of the blood at a centroid 208 of the blood vessel 200. In some cases, the centroid 208 represents 10%, 5%, or 1% of a total area of the cross section of the blood vessel 200. For instance, the velocity at the centroid 208 can be determined based on a Doppler shift of a reflection of an ultrasound signal from the blood crossing the centroid 208. According to some cases, the flow parameter is a flow rate of the blood passing through the cross-section of the blood vessel 200.

In various cases, the blood pressure metrics are based on a change in the flow parameter over the course of the cardiac cycle and/or some average of the flow parameter during the cardiac cycle. For instance, the pulse pressure may be derived based on the change in the flow parameter. In some cases, the MAP is derived based on the mean of the flow parameter.

FIG. 3 illustrates an example pressure waveform 300 that can be generated using various techniques described herein. A horizontal axis represents time and a vertical axis represents pressure in the blood vessel. The waveform 300 of FIG. 3 represents the pressure in a blood vessel over the course of a single cardiac cycle, for instance.

The time axis is divided into two regions representing different phases of the cardiac cycle. A first phase represents systole 302 and a second phase represents diastole 304. It should be noted that the systole 302 represented in FIG. 3 may be slightly delayed with respect to the heart being in systole, and the diastole 304 represented in FIG. 3 may be slightly delayed with respect to the heart being in diastole, depending on the distance between the heart and the blood vessel. The systole 302 phase of FIG. 3 may represent a change in the blood pressure in the blood vessel that occurs due to the systole phase of the heart, and the diastole 304 phase of FIG. 3 may represent a change in the blood pressure in the blood vessel that occurs due to the diastole phase of the heart.

During systole 302, the blood pressure in the blood vessel rises to a peak that represents the systolic pressure 306 of the blood vessel. Subsequently, the pressure decreases until systole 302 ends. During diastole 304, the blood pressure may rise slightly to a second peak that is lower than the systolic pressure 306. Then, the pressure decays to a diastolic pressure 308. A pulse pressure 310 is defined as the difference between the systolic pressure 306 and the diastolic pressure 308. A MAP 312 is defined as the mean of the waveform 300.

The waveform 300, the systolic pressure 306, the diastolic pressure 308, the pulse pressure 310, the MAP 312, or any combination thereof, can be derived based on various width metrics and/or flow parameters of the blood vessel. The width metrics and/or flow parameters can be derived using ultrasound, for instance. In some cases, the waveform 300 is generated based on the following Equation 1:

$$P = {d}_{n} ( t ) * {P}_{p} + {P}_{MAP} \qquad ( 1 )$$

wherein P is the pressure in the blood vessel, dₙ(t) is the normalized distension waveform with respect to time, Pp is the pulse pressure, and P_{MAP} is the MAP.

Various components of Equation 1 can be derived based on the width metrics and/or flow parameters.

In some cases, the normalized distension waveform can be generated using Equation 2A:

$${d}_{1} ( t ) = {d}_{1} ( t ) - \frac{1}{b - α} {\int }_{a}^{b} {d}_{1} ( t ) dt \qquad ( 2 A )$$

wherein a is the time at the beginning of systole, and b is the time at the end of diastole, and d₁(t) is defined according to Equation 2B:

$${d}_{1} ( t ) = \frac{{d}_{m} ( t ) - min ( {d}_{m} ( t ) )}{max ( {d}_{m} ( t ) ) - min ( {d}_{m} ( t ) )} \qquad ( 2 B )$$

wherein dₘ(t) is the measured distension of the blood vessel over the course of the cardiac cycle with respect to time.

In various implementations, the pulse pressure can be generated using Equation 3:

$${P}_{p} = f ( Δ v , PWV ) \qquad ( 3 )$$

wherein Δv is a change in a flow parameter (e.g., blood velocity) through the blood vessel (e.g., through the centroid) during the cardiac cycle, and PWV is the pulse wave velocity of the blood vessel. In some examples, Δv is a difference between the maximum of the flow parameter (e.g., blood velocity) of systole 302 and minimum of the flow parameter at the end of diastole 304.

In some examples, the pulse pressure can be calculated using Equation 4:

$${P}_{p} = f ( Δ v , PWV , s ) \qquad ( 4 )$$

wherein s is a metric representing the size of the subject in which the blood vessel is located. In various cases, s is a metric representative of an amount of fat, muscle, or other soft tissues within the body of the subject, and can be representative of excess weight. In various implementations, s is indicative of a vasculature load of the subject's body.

According to various cases, the MAP can be generated using the following Equation 5:

$${P}_{MAP} = f ( {v}_{mav} , D ) \qquad ( 5 )$$

wherein vₘₐᵥ is the mean of the flow parameter (e.g., blood velocity) through the blood velocity (e.g., through the centroid) during the cardiac cycle, and D is a width metric (e.g., average, such as mean, minimum, or maximum diameter) of the blood vessel.

In some examples, the MAP is further based on the size of the subject, and can be generated using the following Equation 6:

$${P}_{MAP} = f ( {v}_{mav} , D , s ) \qquad ( 6 )$$

It should be noted that various blood pressure metrics described herein can be derived more generally based on a static component of a blood pressure metric (e.g., a component that does not change over the course of the cardiac cycle) as well as a dynamic component of the blood pressure metric (e.g., a component that changes over the course of the cardiac cycle). MAP and pulse pressure are examples of static and dynamic components, but implementations of the present disclosure are not limited to MAP and pulse pressure.

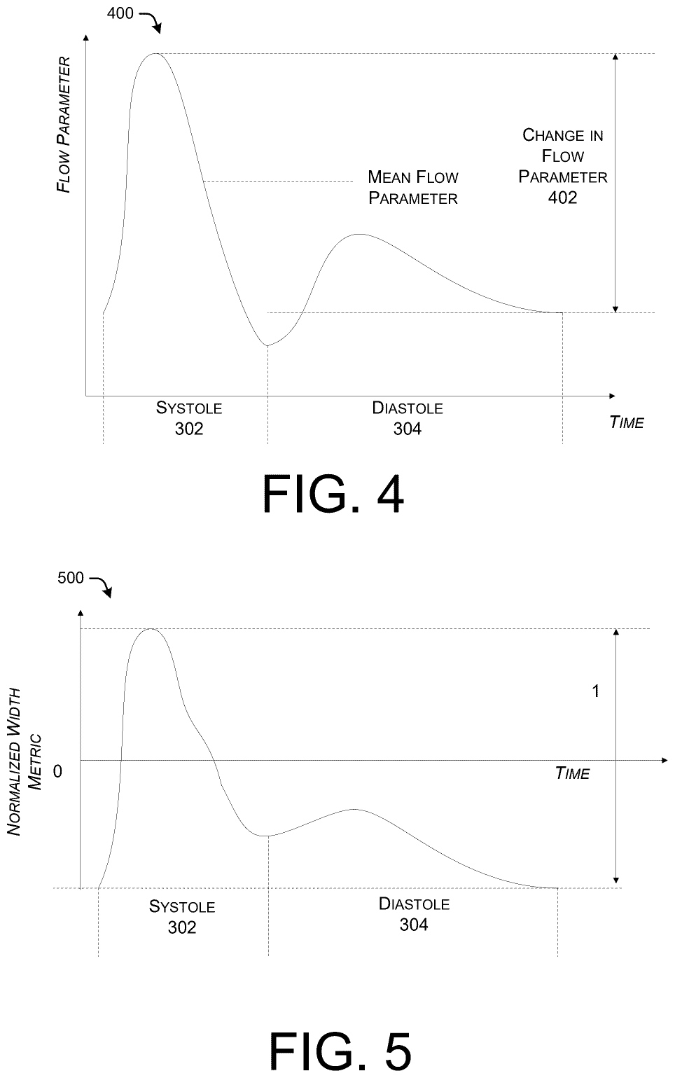

FIG. 4 illustrates an example flow parameter waveform 400 that can be generated using various techniques described herein. The horizontal axis represents time and the vertical axis represents the flow parameter. The flow parameter waveform 400 represents measurements of a flow parameter through a cross-section of a blood vessel (e.g., the blood vessel 110) sampled over a cardiac cycle of a subject in which the blood vessel is located. As shown, the flow parameter waveform 400 varies over the course of the cardiac cycle of the subject, which includes systole 302 and diastole 304. In various implementations, the flow parameter waveform 400 is generated by sampling the flow parameter multiple times over the course of the cardiac cycle. For instance, the flow parameter can be measured using an ultrasound sensor and a Doppler-based analysis.

In various cases, a difference between a maximum and a minimum of the flow parameter waveform 400 is defined as a change in the flow parameter 402. For instance, the change in the flow parameter 402 may be representative of a point at the rise of at the end of diastole and the peak of systole. The minimum of the flow parameter waveform 300, for instance, may occur between systole 302 and diastole 304. In some examples, this value can be utilized to identify the pulse pressure of the subject.

According to some examples, a mean flow parameter 404 can be derived based on the flow parameter waveform 400. For instance, the integral of the flow parameter waveform 400 across the cardiac cycle can be calculated and divided by the duration of the cardiac cycle. According to various implementations, the mean flow parameter can be used to estimate the MAP of the subject.

FIG. 5 illustrates an example of a normalized width metric waveform 500 that can be generated using various techniques described herein. The horizontal axis represents time and the vertical axis represents the normalized width metric. In various cases, the width metric is of a blood vessel. For instance, a waveform representing the width metric can be generated by real-time imaging a cross-section of the blood vessel multiple times over the course of a cardiac cycle. In some cases, the width metric is generated by measuring the change of the width metric directly. Various image processing techniques described herein can be utilized to identify the width metric based on the cross-sectional images of the blood vessel. In various cases, the normalized width metric waveform 500 can be generated by applying the width metric waveform to Equations 2A and 2B. As shown, the normalized width metric waveform 500 is centered at 0. That is, a mean of the normalized width metric waveform 500 is equal to 0. Further, a difference between the minimum and the maximum of the normalized width metric waveform 500 is 1. In various implementations, the normalized width metric waveform 500 can be utilized to derive a blood pressure waveform.

In some implementations, the width metric waveform 500 is altered such that the horizontal axis is at the minimum at the end of diastole of the width metric waveform 500 and the maximum of the width metric waveform is 1. In this case, the blood pressure waveform can be generated by multiplying pulse pressure and adding diastolic blood pressure, instead of MAP. For instance, the following Equation 7 can be utilized to determine the normalized distension in these instances:

$${d}_{n} ( t ) = \frac{{d}_{m} ( t ) - min ( {d}_{m} ( t ) )}{max ( {d}_{m} ( t ) ) - min ( {d}_{m} ( t ) )} \qquad ( 7 )$$

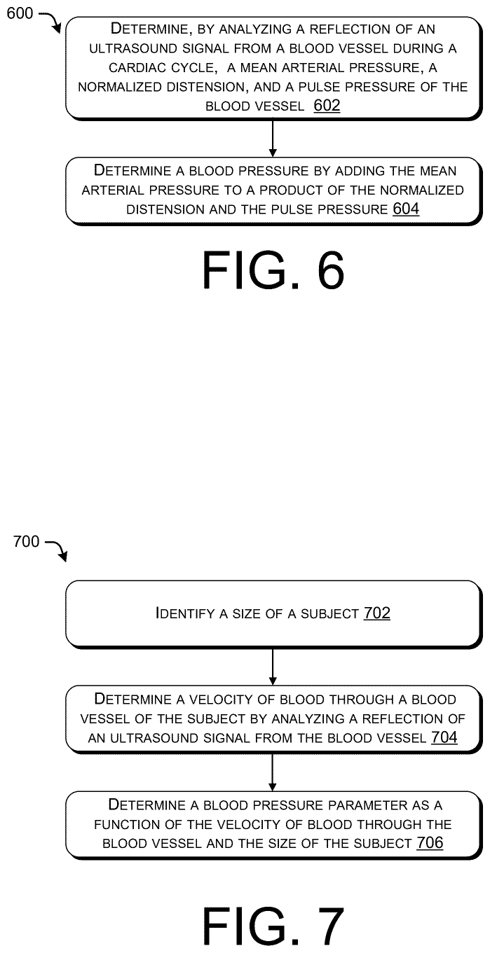

FIG. 6 illustrates an example process 600 for noninvasively identifying the blood pressure of a subject using ultrasound. The process 600, in various examples, is performed by an entity including the blood pressure monitor 104, the monitor-defibrillator 130, an ultrasound monitor, a medical device, one or more processors, a computing device, or any combination thereof.

At 602, the entity determines, by analyzing a reflection of an ultrasound signal from a blood vessel during a cardiac cycle, a mean arterial pressure, a normalized distension, and a pulse pressure of the blood vessel. In some examples, the entity outputs the ultrasound signal toward the blood vessel during the cardiac cycle. For instance, the entity includes one or more transducers configured to output the ultrasound signal and/or to detect the reflection of the ultrasound signal. According to some examples, the entity selectively outputs the ultrasound signal and/or detects the reflection of the ultrasound signal when it determines that it has less than a threshold acceleration (e.g., the entity is not significantly moving).

In some cases, the blood pressure is an artery. The entity, for instance, determines the mean arterial pressure by determining a mean of the velocity of blood through the artery (e.g., over the course of the cardiac cycle). The velocity of the blood can be determined using a Doppler- or speckle-based analysis of the reflection of the ultrasound signal from the blood in the blood vessel. In some cases, the entity determines a diameter of the artery by analyzing the reflection. In various examples, the entity determines the mean arterial pressure based on the mean of the velocity of the blood and the diameter of the artery. For instance, the entity identifies the mean arterial pressure by identifying an entry of a look-up table corresponding to the mean of the velocity of the blood and the diameter of the artery, or by calculating the mean arterial pressure as a function of the mean of the velocity of the blood and the diameter of the artery. In some cases, the mean arterial pressure is further based on a size of the subject.

In some examples, the entity determines a width metric of the blood vessel by analyzing the reflection of the ultrasound signal. For instance, the width metric includes a diameter of the blood vessel, a radius of the blood vessel, or a cross-sectional area of the blood vessel. The entity, for instance, determines a waveform representing the width metric over the course of the cardiac cycle. In some cases, the entity normalizes the width metric by dividing the width metric by a maximum change of the width metric during the cardiac cycle and subtracting its diastolic minimum. In some examples, the entity further determines the normalized distension by subtracting the mean of the normalized width metric from the normalized width metric. In other examples, the entity further determines the normalized distension by subtracting the minimum at the end of diastole of the width metric (e.g., wherein a mean of the normalized distension is zero or a minimum of the normalized distension is zero).

In various examples, the pulse pressure of the subject can be derived by determining a change of the velocity of the blood through the blood vessel between a systolic phase and a diastolic phase of the cardiac cycle. Further, the entity may determine a pulse wave velocity of the blood vessel. According to various cases, the entity may derive the pulse pressure by identifying an entry of a look-up table corresponding to the change in the velocity of the blood through the blood vessel and the pulse wave velocity, or as a function of the change in the velocity and the pulse wave velocity.

At 604, the entity determines a blood pressure by adding the mean arterial pressure to a product of the normalized distension and the pulse pressure. Alternatively, the entity determines a blood pressure by adding the diastolic pressure to a product of the normalized distension and the pulse pressure. In various cases, the entity further outputs an indication of the blood pressure. The blood pressure may be output as a waveform and/or as a numeric value. In some cases, the entity determines and outputs an indication (e.g., a numeric indicator, or some other indicator such as a color change on a display) of whether the blood pressure is increasing or decreasing. According to some examples, the entity further estimates the blood pressure using an alternative technique in addition to the ultrasound-based technique. For instance, the entity may further perform an additional estimation of the blood pressure using a blood pressure cuff. The entity may determine that one estimate of the blood pressure is more accurate than the other, and selectively output the most accurate estimation of the blood pressure.

FIG. 7 illustrates an example process 700 for noninvasively determining a blood pressure parameter of a subject using ultrasound. The process 700, in various examples, is performed by an entity including the blood pressure monitor 104, the monitor-defibrillator 130, an ultrasound monitor, a medical device, one or more processors, a computing device, or any combination thereof.

At 702, the entity identifies a size of a subject. Various techniques can be utilized to identify the size of the subject. In some cases, the entity itself includes a sensor configured to detect a height and/or a weight of the subject. In some examples, the entity receives, from an EMR device, a signal indicating the size of the subject. In some examples, the entity receives the signal indicating the size of the subject from a user. For example, a rescuer can input an estimated size of the subject into the entity.

At 704, the entity determines a velocity of blood through a blood vessel of the subject by analyzing a reflection of an ultrasound signal from the blood vessel. In some examples, the entity outputs the ultrasound signal toward the blood vessel during the cardiac cycle. For instance, the entity includes one or more transducers configured to output the ultrasound signal and/or to detect the reflection of the ultrasound signal. According to some examples, the entity selectively outputs the ultrasound signal and/or detects the reflection of the ultrasound signal when it determines that it has less than a threshold movement with respect to the blood vessel (e.g., velocity, acceleration, etc.). The movement, for instance, is detected by a motion sensor (e.g., accelerometer) or by analyzing the reflection of the ultrasound signal itself (e.g., by analyzing images generated based on the reflection).

The entity, in some examples, performs a Doppler-based technique for determining the velocity of the blood through the blood vessel. For example, the entity may determine a Doppler shift between the ultrasound signal, as transmitted, and the reflection of the ultrasound signal from the blood in the blood vessel. The velocity of the blood through the blood vessel is derived, for instance, as a function of the Doppler shift. The Doppler shift, in some cases, represents a frequency and/or phase shift.

At 706, the entity determines a blood pressure parameter as a function of the velocity of the blood through the blood vessel and the size of the subject. Optionally, the entity outputs an indication of the blood pressure parameter. In some cases, the blood pressure parameter is further determined based on a pulse wave velocity of the blood vessel and/or a width metric of the blood vessel.

According to some examples, the velocity determined in 704 is a difference between the velocity of the blood during a systolic phase of the cardiac cycle and the velocity of the blood during (or at the end of) a diastolic phase of the cardiac cycle. In various cases, the blood pressure parameter derived based on the difference is a pulse pressure of the subject.

In some cases, the velocity determined in 704 is a mean of the velocity of the blood through the blood vessel across the cardiac cycle. For instance, the blood pressure parameter determined based on the mean of the velocity is a mean arterial pressure of the subject.

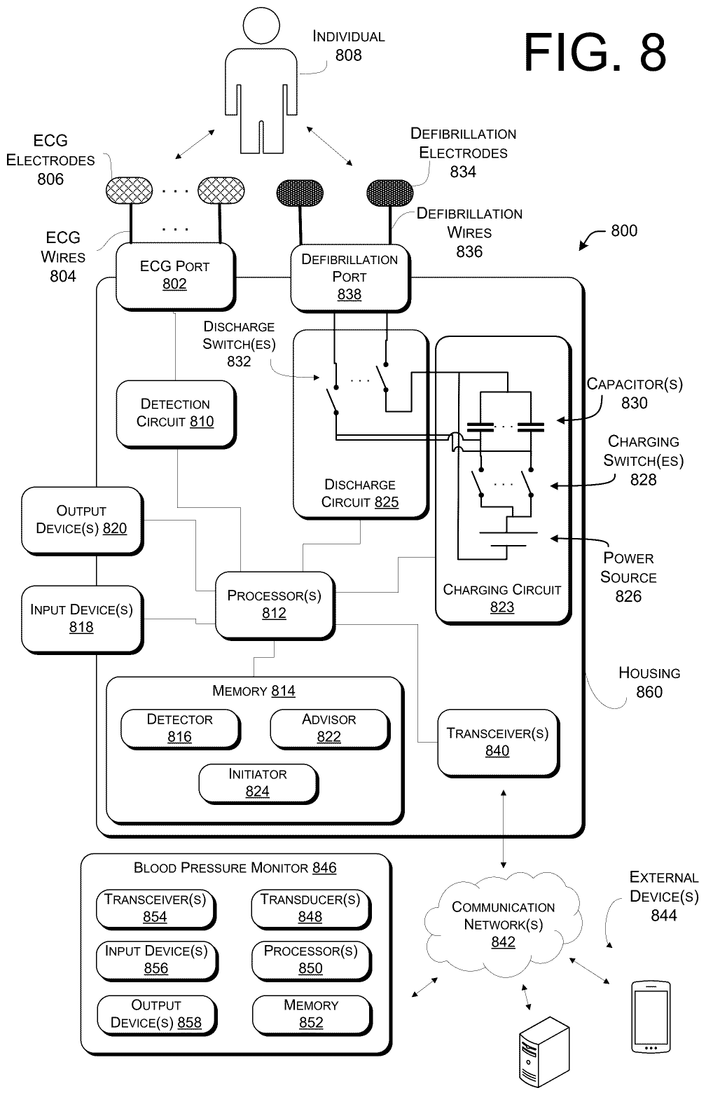

FIG. 8 illustrates an example of an external defibrillator 800 configured to perform various functions described herein. For example, the external defibrillator 800 is the monitor-defibrillator 130 described above with reference to FIG. 1.

The external defibrillator 800 includes an ECG port 802 connected to multiple ECG wires 804. In some cases, the ECG wires 804 are removeable from the ECG port 802. For instance, the ECG wires 804 are plugged into the ECG port 802 via connectors. The ECG wires 804 are connected to ECG electrodes 806, respectively. In various implementations, the ECG electrodes 806 are disposed on different locations on an individual 808. A detection circuit 810 is configured to detect relative voltages between the ECG electrodes 806. These voltages are indicative of the electrical activity of the heart of the individual 808.

In various implementations, the ECG electrodes 806 are in contact with the different locations on the skin of the individual 808. In some examples, a first one of the ECG electrodes 806 is placed on the skin between the heart and right arm of the individual 808, a second one of the ECG electrodes 806 is placed on the skin between the heart and left arm of the individual 808, and a third one of the ECG electrodes 806 is placed on the skin between the heart and a leg (either the left leg or the right leg) of the individual 808. In these examples, the detection circuit 810 is configured to measure the relative voltages between the first, second, and third ECG electrodes 806. Respective pairings of the ECG electrodes 806 are referred to as “leads,” and the voltages between the pairs of ECG electrodes 806 are known as “lead voltages.” In some examples, more than three ECG electrodes 806 are included, such that 5-lead or 12-lead ECG signals are detected by the detection circuit 810.

The detection circuit 810 includes at least one analog circuit, at least one digital circuit, or a combination thereof. The detection circuit 810 receives the analog electrical signals from the ECG electrodes 806, via the ECG port 802 and the ECG wires 804. In some cases, the detection circuit 810 includes one or more analog filters configured to filter noise and/or artifact from the electrical signals. The detection circuit 810 includes an analog-to-digital (ADC) in various examples. The detection circuit 810 generates a digital signal indicative of the analog electrical signals from the ECG electrodes 806. This digital signal can be referred to as an “ECG signal” or an “ECG.”

In some cases, the detection circuit 810 further detects an electrical impedance between at least one pair of the ECG electrodes 806. For example, the detection circuit 810 includes, or otherwise controls, a power source that applies a known voltage (or current) across a pair of the ECG electrodes 806 and detects a resultant current (or voltage) between the pair of the ECG electrodes 806. The impedance is generated based on the applied signal (voltage or current) and the resultant signal (current or voltage). In various cases, the impedance corresponds to respiration of the individual 808, chest compressions performed on the individual 808, and other physiological states of the individual 808. In various examples, the detection circuit 810 includes one or more analog filters configured to filter noise and/or artifact from the resultant signal. The detection circuit 810 generates a digital signal indicative of the impedance using an ADC. This digital signal can be referred to as an “impedance signal” or an “impedance.”

The detection circuit 810 provides the ECG signal and/or the impedance signal one or more processors 812 in the external defibrillator 800. In some implementations, the processor(s) 812 includes a central processing unit (CPU), a graphics processing unit (GPU), both CPU and GPU, or other processing unit or component known in the art.

The processor(s) 812 is operably connected to memory 814. In various implementations, the memory 814 is volatile (such as random access memory (RAM)), non-volatile (such as read only memory (ROM), flash memory, etc.) or some combination of the two. The memory 814 stores instructions that, when executed by the processor(s) 812, causes the processor(s) 812 to perform various operations. In various examples, the memory 814 stores methods, threads, processes, applications, objects, modules, any other sort of executable instruction, or a combination thereof. In some cases, the memory 814 stores files, databases, or a combination thereof. In some examples, the memory 814 includes, but is not limited to, RAM, ROM, electrically erasable programmable read-only memory (EEPROM), flash memory, or any other memory technology. In some examples, the memory 814 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 812 and/or the external defibrillator 800. In some cases, the memory 814 at least temporarily stores the ECG signal and/or the impedance signal.

In various examples, the memory 814 includes a detector 816, which causes the processor(s) 812 to determine, based on the ECG signal and/or the impedance signal, whether the individual 808 is exhibiting a particular heart rhythm. For instance, the processor(s) 812 determines whether the individual 808 is experiencing a shockable rhythm that is treatable by defibrillation. Examples of shockable rhythms include ventricular fibrillation (VF) and pulseless ventricular tachycardia (VT). In some examples, the processor(s) 812 determines whether any of a variety of different rhythms (e.g., asystole, sinus rhythm, atrial fibrillation (AF), etc.) are present in the ECG signal.

The processor(s) 812 is operably connected to one or more input devices 818 and one or more output devices 820. Collectively, the input device(s) 818 and the output device(s) 820 function as an interface between a user and the defibrillator 800. The input device(s) 818 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 820 includes at least one of a display, a speaker, a haptic output device, a printer, or any combination thereof.

In various examples, the processor(s) 812 causes a display among the input device(s) 818 to visually output a waveform of the ECG signal and/or the impedance signal. In some implementations, the input device(s) 818 includes one or more touch sensors, the output device(s) 820 includes a display screen, and the touch sensor(s) are integrated with the display screen. Thus, in some cases, the external defibrillator 800 includes a touchscreen configured to receive user input signal(s) and visually output physiological parameters, such as the ECG signal and/or the impedance signal.

In some examples, the memory 814 includes an advisor 822, which, when executed by the processor(s) 812, causes the processor(s) 812 to generate advice and/or control the output device(s) 820 to output the advice to a user (e.g., a rescuer). In some examples, the processor(s) 812 provides, or causes the output device(s) 820 to provide, an instruction to perform CPR on the individual 808. In some cases, the processor(s) 812 evaluates, based on the ECG signal, the impedance signal, or other physiological parameters (e.g., one or more blood pressure parameters), CPR being performed on the individual 808 and causes the output device(s) 820 to provide feedback about the CPR in the instruction. According to some examples, the processor(s) 812, upon identifying that a shockable rhythm is present in the ECG signal, causes the output device(s) 820 to output an instruction and/or recommendation to administer a defibrillation shock to the individual 808.

The memory 814 also includes an initiator 824 which, when executed by the processor(s) 812, causes the processor(s) 812 to control other elements of the external defibrillator 800 in order to administer a defibrillation shock to the individual 808. In some examples, the processor(s) 812 executing the initiator 824 selectively causes the administration of the defibrillation shock based on determining that the individual 808 is exhibiting the shockable rhythm and/or based on an input from a user (received, e.g., by the input device(s) 818. In some cases, the processor(s) 812 causes the defibrillation shock to be output at a particular time, which is determined by the processor(s) 812 based on the ECG signal and/or the impedance signal.

The processor(s) 812 is operably connected to a charging circuit 823 and a discharge circuit 825. In various implementations, the charging circuit 823 includes a power source 826, one or more charging switches 828, and one or more capacitors 830. The power source 826 includes, for instance, a battery. The processor(s) 812 initiates a defibrillation shock by causing the power source 826 to charge at least one capacitor among the capacitor(s) 830. For example, the processor(s) 812 activates at least one of the charging switch(es) 828 in the charging circuit 823 to complete a first circuit connecting the power source 826 and the capacitor to be charged. Then, the processor(s) 812 causes the discharge circuit 825 to discharge energy stored in the charged capacitor across a pair of defibrillation electrodes 834, which are in contact with the individual 808. For example, the processor(s) 812 deactivates the charging switch(es) 828 completing the first circuit between the capacitor(s) 830 and the power source 826, and activates one or more discharge switches 832 completing a second circuit connecting the charged capacitor 830 and at least a portion of the individual 808 disposed between defibrillation electrodes 834.

The energy is discharged from the defibrillation electrodes 834 in the form of a defibrillation shock. For example, the defibrillation electrodes 834 are connected to the skin of the individual 808 and located at positions on different sides of the heart of the individual 808, such that the defibrillation shock is applied across the heart of the individual 808. The defibrillation shock, in various examples, depolarizes a significant number of heart cells in a short amount of time. The defibrillation shock, for example, interrupts the propagation of the shockable rhythm (e.g., VF or VT) through the heart. In some examples, the defibrillation shock is 200 J or greater with a duration of about 0.015 seconds. In some cases, the defibrillation shock has a multiphasic (e.g., biphasic) waveform. The discharge switch(es) 832 are controlled by the processor(s) 812, for example. In various implementations, the defibrillation electrodes 834 are connected to defibrillation wires 836. The defibrillation wires 836 are connected to a defibrillation port 838, in implementations. According to various examples, the defibrillation wires 836 are removable from the defibrillation port 838. For example, the defibrillation wires 836 are plugged into the defibrillation port 838.

In various implementations, the processor(s) 812 is operably connected to one or more transceivers 840 that transmit and/or receive data over one or more communication networks 842. For example, the transceiver(s) 840 includes a network interface card (NIC), a network adapter, a local area network (LAN) adapter, or a physical, virtual, or logical address to connect to the various external devices and/or systems. In various examples, the transceiver(s) 840 includes any sort of wireless transceivers capable of engaging in wireless communication (e.g., radio frequency (RF) communication). For example, the communication network(s) 842 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 840 includes other wireless modems, such as a modem for engaging in WI-FI®, WIGIG®, WIMAX®, BLUETOOTH®, or infrared communication over the communication network(s) 842.

The defibrillator 800 is configured to transmit and/or receive data (e.g., ECG data, impedance data, data indicative of one or more detected heart rhythms of the individual 808, data indicative of one or more defibrillation shocks administered to the individual 808, etc.) with one or more external devices 844 via the communication network(s) 842. The external devices 844 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 842. In some examples, the external device(s) 844 is located remotely from the defibrillator 800, such as at a remote clinical environment (e.g., a hospital). According to various implementations, the processor(s) 812 causes the transceiver(s) 840 to transmit data to the external device(s) 844. In some cases, the transceiver(s) 840 receives data from the external device(s) 844 and the transceiver(s) 840 provide the received data to the processor(s) 812 for further analysis.

According to various cases, the external device(s) 844 include a blood pressure monitor 846 configured to non-invasively detect a blood pressure of the individual 808. In some examples, the blood pressure monitor 846 is the blood pressure monitor 104 described above with reference to FIG. 1. The blood pressure monitor 846 includes at least one transducer 848 configured to transmit ultrasound into the body of the individual 808. In addition, the transducer(s) 848 is configured to detect reflections of the ultrasound from physiological structures in the body of the individual 808, such as a blood vessel.

The blood pressure monitor 846, in various cases, includes at least one processor 850 configured to execute instructions stored in memory 852, thereby performing various functions described herein. In some examples, the processor(s) 850 is configured to analyze the reflections of the ultrasound detected by the transducer(s) 848 in order to determine a width metric, blood velocity, blood parameter metric, or any other metric described herein, of the individual 808. Any type of processor and/or memory described elsewhere herein can be suitable as the processor(s) 850 and/or memory 852 of the blood pressure monitor 846.

In various cases, the blood pressure monitor 846 further includes one or more transceivers 854 configured to transmit and/or receive communication signals with the transceiver(s) 840 of the defibrillator 800 over the communication network(s) 842. In some examples, the blood pressure monitor 846 further includes at least one input device 856 (e.g., a button, a touchscreen, a microphone, etc.) and at least one output device 858 (e.g., a display, a speaker, etc.). Any type of input device and/or output device described herein can be included in the input device(s) 856 and/or output device(s) 858. In some examples, the input device(s) 856 includes an accelerometer and/or an additional blood pressure sensor (e.g., a blood pressure cuff, pressure gauge, oscillometric blood pressure sensor, or any combination thereof).

In particular examples, the blood pressure monitor 846 transmits an indication of a blood pressure of the individual 808 to the defibrillator 800 via a communication signal. In some cases, the defibrillator 800 performs various functions based on the blood pressure reported by the blood pressure monitor 846. For example, the detector 816 may include instructions to detect pulseless VT in response to detecting VT in the ECG of the individual 808 as well as in response to determining that the blood pressure detected by the blood pressure monitor 846 is below a threshold blood pressure. In various cases, the advisor 822 includes instructions that cause the processor(s) 812 to output a recommendation to administer an electrical shock to the individual 808 in response to detecting the pulseless VT. In some cases, the output device(s) 820 of the defibrillator 800 output an indication of the blood pressure detected by the blood pressure monitor 846.

In various implementations, the external defibrillator 800 also includes a housing 860 that at least partially encloses other elements of the external defibrillator 800. For example, the housing 860 encloses the detection circuit 810, the processor(s) 812, the memory 814, the charging circuit 823, the transceiver(s) 840, or any combination thereof. In some cases, the input device(s) 818 and output device(s) 820 extend from an interior space at least partially surrounded by the housing 860 through a wall of the housing 860. In various examples, the housing 860 acts as a barrier to moisture, electrical interference, and/or dust, thereby protecting various components in the external defibrillator 800 from damage.

In some implementations, the external defibrillator 800 is an automated external defibrillator (AED) operated by an untrained user (e.g., a bystander, layperson, etc.) and can be operated in an automatic mode. In automatic mode, the processor(s) 812 automatically identifies a rhythm in the ECG signal, makes a decision whether to administer a defibrillation shock, charges the capacitor(s) 830, discharges the capacitor(s) 830, or any combination thereof. In some cases, the processor(s) 812 controls the output device(s) 820 to output (e.g., display) a simplified user interface to the untrained user. For example, the processor(s) 812 refrains from causing the output device(s) 820 to display a waveform of the ECG signal and/or the impedance signal to the untrained user, in order to simplify operation of the external defibrillator 800.

In some examples, the external defibrillator 800 is a monitor-defibrillator utilized by a trained user (e.g., a clinician, an emergency responder, etc.) and can be operated in a manual mode or the automatic mode. When the external defibrillator 800 operates in manual mode, the processor(s) 812 cause the output device(s) 820 to display a variety of information that may be relevant to the trained user, such as waveforms indicating the ECG data and/or impedance data, notifications about detected heart rhythms, and the like.

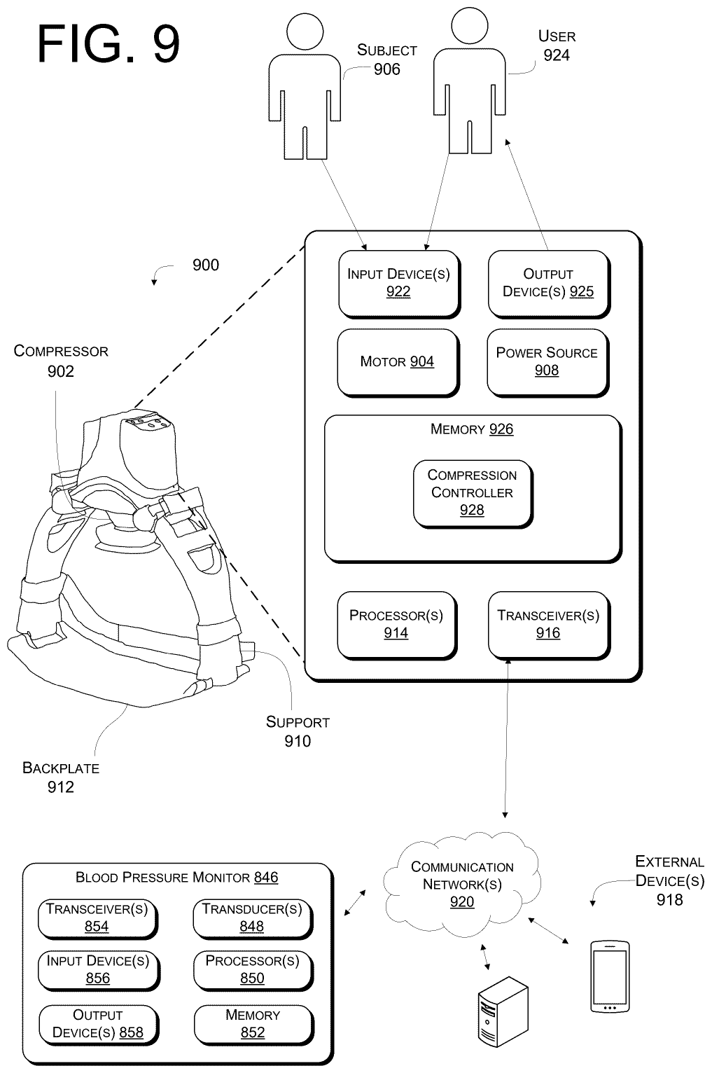

FIG. 9 illustrates a chest compression device 900 configured to perform various functions described herein. For example, the chest compression device 900 may be communicatively coupled with the blood pressure monitor 104 described above with reference to FIG. 1.

In various implementations, the chest compression device 900 includes a compressor 902 that is operatively coupled to a motor 904. The compressor 902 physically administers a force to the chest of a subject 906 that compresses the chest of the subject 906. In some examples, the compressor 902 includes at least one piston that periodically moves between two positions (e.g., a compressed position and a release position) at a compression frequency. For example, when the piston is positioned on the chest of the subject 906, the piston compresses the chest when the piston is moved into the compressed position. A suction cup may be positioned on a tip of the piston, such that the suction cup contacts the chest of the subject 906 during operation. In various cases, the compressor 902 includes a band that periodically tightens to a first tension and loosens to a second tension at a compression frequency. For instance, when the band is disposed around the chest of the subject 906, the band compresses the chest when the band tightens.

The motor 904 is configured to convert electrical energy stored in a power source 908 into mechanical energy that moves and/or tightens the compressor 902, thereby causing the compressor 902 to administer the force to the chest of the subject 906. In various implementations, the power source 908 is portable. For instance, the power source 908 includes at least one rechargeable (e.g., lithium-ion) battery. In some cases, the power source 908 supplies electrical energy to one or more elements of the chest compression device 900 described herein.

In various cases, the chest compression device 900 includes a support 910 that is physically coupled to the compressor 902, such that the compressor 902 maintains a position relative to the subject 906 during operation. In some implementations, the support 910 is physically coupled to a backplate 912, cot, or other external structure with a fixed position relative to the subject 906. According to some cases, the support 910 is physically coupled to a portion of the subject 906, such as wrists of the subject 906.

The operation of the chest compression device 900 may be controlled by at least one processor 914. In various implementations, the motor 904 is communicatively coupled to the processor(s) 914. Specifically, the processor(s) 914 is configured to output a control signal to the motor 904 that causes the motor 904 to actuate the compressor 902. For instance, the motor 904 causes the compressor 902 to administer the compressions to the subject 906 based on the control signal. In some cases, the control signal indicates one or more treatment parameters of the compressions. Examples of treatment parameters include a frequency, timing, depth, force, position, velocity, and acceleration of the compressor 902 administering the compressions. According to various cases, the control signal causes the motor 904 to cease compressions.

In various implementations, the chest compression device 900 includes at least one transceiver 916 configured to communicate with at least one external device 918 over one or more communication networks 920. Any communication network described herein can be included in the communication network(s) 920 illustrated in FIG. 9. The external device(s) 918, for example, includes at least one of a monitor-defibrillator, an AED, an ECMO device, a ventilation device, a patient monitor, a mobile phone, a server, or a computing device. In some implementations, the transceiver(s) 916 is configured to communicate with the external device(s) 918 by transmitting and/or receiving signals wirelessly. For example, the transceiver(s) 916 includes a NIC, a network adapter, a LAN adapter, or a physical, virtual, or logical address to connect to the various external devices and/or systems. In various examples, the transceiver(s) 916 includes any sort of wireless transceivers capable of engaging in wireless communication (e.g., RF communication). For example, the communication network(s) 920 includes one or more wireless networks that include a 3GPP network, such as an LTE RAN (e.g., over one or more LTE bands), an NR RAN (e.g., over one or more NR bands), or a combination thereof. In some cases, the transceiver(s) 916 includes other wireless modems, such as a modem for engaging in WI-FI®, WIGIG®, WIMAX®, BLUETOOTH®, or infrared communication over the communication network(s) 920. The signals, in various cases, encode data in the form of data packets, datagrams, or the like. In some cases, the signals are transmitted as compressions are being administered by the chest compression device 900 (e.g., for real-time feedback by the external device(s) 918), after compressions are administered by the chest compression device 900 (e.g., for post-event review at the external device 918), or a combination thereof.

In various examples, the external device(s) 918 include the blood pressure monitor 846 described above with reference to FIG. 8. For instance, the blood pressure monitor 846 may transmit, to the chest compression device 900, a communication signal indicating one or more blood pressure parameters. In some cases, the chest compression device 900 may transmit, to the blood pressure monitor 846, an indication of the size of the subject 906. For instance, the size of the subject 906 may be derived by the chest compression device 900 based on a position of the compressor 902 during the administration of chest compressions.

In various cases, the processor(s) 914 generates the control signal based on data encoded in the signals received from the external device(s) 918. For instance, the signals include an instruction to initiate the compressions, and the processor(s) 914 instructs the motor 904 to begin actuating the compressor 902 in accordance with the signals.

In some cases, the chest compression device 900 includes at least one input device 922. In various examples, the input device(s) 922 is configured to receive an input signal from a user 924, who may be a rescuer treating the subject 906. Examples of the input device(s) 922 include, for instance, at a keypad, a cursor control, a touch-sensitive display, a voice input device (e.g., a microphone), a haptic feedback device (e.g., a gyroscope), or any combination thereof. In various implementations, the processor(s) 914 generate the control signal based on the input signal. For instance, the processor(s) 914 generate the control signal to adjust a frequency of the compressions based on the chest compression device 900 detecting a selection by the user 924 of a user interface element displayed on a touchscreen or detecting the user 924 pressing a button integrated with an external housing of the chest compression device 900.

According to some examples, the input device(s) 922 include one or more sensors. The sensor(s), for example, is configured to detect a physiological parameter of the subject 906. In some implementations, the sensor(s) is configured to detect a state parameter of the chest compression device 900, such as a position of the compressor 902 with respect to the subject 906 or the backplate 912, a force administered by the compressor 902 on the subject 906, a force administered onto the backplate 912 by the body of the subject 906 during a compression, or the like. According to some implementations, the signals transmitted by the transceiver(s) 916 indicate the physiological parameter(s) and/or the state parameter(s).

The chest compression device 900 further includes at least one output device 925, in various implementations. Examples of the output device(s) 925 include, for instance, least one of a display (e.g., a projector, an LED screen, etc.), a speaker, a haptic output device, a printer, or any combination thereof. In some implementations, the output device(s) 925 include a screen configured to display various parameters detected by and/or reported to the chest compression device 900, a charge level of the power source 908, a timer indicating a time since compressions were initiated or paused, and other relevant information.

The chest compression device 900 further includes memory 926. In various implementations, the memory 926 is volatile (such as random access memory (RAM)), non-volatile (such as read only memory (ROM), flash memory, etc.) or some combination of the two.

The memory 926 stores instructions that, when executed by the processor(s) 914, causes the processor(s) 914 to perform various operations. In various examples, the memory 926 stores methods, threads, processes, applications, objects, modules, any other sort of executable instruction, or a combination thereof. In some cases, the memory 926 stores files, databases, or a combination thereof. In some examples, the memory 926 includes, but is not limited to, RAM, ROM, EEPROM, flash memory, or any other memory technology. In some examples, the memory 926 includes one or more of CD-ROMs, DVDs, CAM, or other optical storage, magnetic cassettes, magnetic tape, magnetic disk storage or other magnetic storage devices, or any other medium which can be used to store the desired information. In various cases, the memory 926 stores instructions, programs, threads, objects, data, or any combination thereof, that cause the processor(s) 914 to perform various functions. In various cases, the memory 926 stores one or more parameters that are detected by the chest compression device 900 and/or reported to the chest compression device 900.

In implementations of the present disclosure, the memory 926 also stores instructions for executing a compression controller 928. When executed by the processor(s) 914, the compression controller 928 may initiate, pause, or modify the chest compressions administered by the chest compression device 900 to the subject 906. In some cases, the compression controller 928 includes instructions for initiating and/or pausing the chest compressions based on the indication of the blood pressure reported by the blood pressure monitor 846. For example, the compression controller 928, when executed by the processor(s) 914, may cause the chest compression device 900 to pause chest compressions in response to determining that the blood pressure is above a first threshold associated with spontaneous circulation. In some cases, the chest compression controller 928, when executed by the processor(s) 914, may cause the chest compression device 900 to initiate chest compressions, or to change a chest compression parameter (e.g., chest compression depth, duty cycle, position, etc.) in response to determining that the blood pressure is below a second threshold associated with insufficient circulation.

### EXAMPLE CLAUSES

The following Clauses provide various examples of the present disclosure:

1. A blood pressure monitor, including: an ultrasound transducer configured to: generate an ultrasound signal; and detect a reflection of the ultrasound signal from an artery of a subject during a cardiac cycle of the subject; a display; and a processor configured to: determine a velocity of blood through the artery of the subject during the cardiac cycle by analyzing the reflection of the ultrasound signal; determine a distension waveform of the artery of the subject during the cardiac cycle by analyzing the reflection of the ultrasound signal; determine a pulse pressure of the subject by: determining a change in the velocity of blood through the artery of the subject between a systolic phase and a diastolic phase of the cardiac cycle; determining a pulse wave velocity of blood of the artery of the subject; determine a mean arterial pressure of the subject by: determining an average of the velocity of the blood through the artery of the subject during the cardiac cycle; and determining an average of the distension waveform of the artery of the subject during the cardiac cycle; determine a normalized distension waveform of the artery during the cardiac cycle by: subtracting, from the distension waveform of the artery during the cardiac cycle, a minimum of the distension waveform of the artery during the cardiac cycle; and in response to subtracting the minimum of the distension waveform of the artery, generating an intermediary waveform by dividing the distension waveform of the artery during the cardiac cycle by a maximum change in the distension waveform of the artery during the cardiac cycle; and subtracting, from the intermediary waveform, the average of the distension waveform of the artery during the cardiac cycle; and determine a blood pressure waveform of the subject by adding, to a product of the normalized distension waveform and the pulse pressure, the mean arterial pressure; and cause the display to visually present the blood pressure waveform of the subject.

2. The blood pressure monitor of clause 1, wherein the processor is configured to determine the pulse pressure of the subject by: determining a vasculature load of the subject as a function of a size of the subject, a height of the subject, a weight of the subject, a body fat percentage, a height-weight ratio of the subject, or a body mass index (BMI) of the subject; and determining the pulse pressure by: identifying an entry of a look-up table corresponding to the change in the velocity of blood through the artery, the pulse wave velocity, and the vasculature load; or calculating the pulse pressure as a function of the change in the velocity of blood through the artery, the pulse wave velocity, and the vasculature load.

3. The blood pressure monitor of clause 2, the look-up table being a first look-up table, wherein the processor is configured to determine the mean arterial pressure of the subject by: identifying an entry of a second look-up table corresponding to the mean of the velocity through the artery during the cardiac cycle, the mean of the distension waveform of the artery during the cardiac cycle, and the vasculature load; or calculating the mean arterial pressure of the subject as a function of the mean of the velocity through the artery during the cardiac cycle, the mean of the distension of the artery during the cardiac cycle, and the vasculature load.

4. A medical device, including: an ultrasound transducer configured to: output, toward a blood vessel of a subject during a cardiac cycle of the subject, an ultrasound signal; and detect, from the blood vessel of the subject during the cardiac cycle of the subject, a reflection of the ultrasound signal; and a processor configured to: determine a normalized distension of the blood vessel of the subject during the cardiac cycle by analyzing the reflection of the ultrasound signal; determine a blood pressure of the subject by adding a mean arterial pressure of the subject to a product of the normalized distension of the blood vessel and a pulse pressure of the subject; and output an indication of the blood pressure.

5. The medical device of clause 4, wherein the processor is configured to determine the normalized distension of the blood vessel of the subject during the cardiac cycle by: determining, by analyzing the reflection of the ultrasound signal, a width metric of the blood vessel during the cardiac cycle, the width metric including a diameter of the blood vessel, a radius of the blood vessel, or a cross-sectional area of the blood vessel; determining a normalized width metric of the blood vessel during the cardiac cycle by dividing the width metric of the blood vessel during the cardiac cycle by a maximum change of the width metric of the blood vessel during the cardiac cycle; and determining the normalized distension of the blood vessel by subtracting the mean of the width metric of the blood vessel during the cardiac cycle from the normalized width metric.

6. The medical device of clause 4 or 5, the blood vessel including an artery, wherein the processor is further configured to determine the mean arterial pressure of the subject by: determining a mean of a velocity of blood through the artery by analyzing the reflection of the ultrasound signal; determining a diameter of the artery by analyzing the reflection of the ultrasound signal; and determining the mean arterial pressure of the subject by: identifying an entry of a look-up table corresponding to the mean of the velocity of blood through the artery and the diameter of the artery; or calculating the mean arterial pressure of the subject as a function of the mean of the velocity of blood through the artery and the diameter of the artery.

7. The medical device of clause 6, wherein determining the mean of the velocity of blood through the artery includes: determining a Doppler shift by identifying a difference between a frequency or phase of the ultrasound signal and a frequency or phase of the reflection of the ultrasound signal, the reflection of the ultrasound signal being from blood disposed in the artery; and determining the velocity of blood through the artery as a function of the Doppler shift.

8. The medical device of clause 6 or 7, wherein the processor is further configured to: identifying a size of the subject, wherein the entry of the look-up table further corresponds to the size of the subject or the function is further dependent on the size of the subject.

9. The medical device of clause 8, wherein the size of the subject includes a body mass index (BMI) of the subject, a body fat percentage, or a clothing size of the subject.

10. The medical device of any of clauses 4 to 9, wherein the processor is further configured to: determine the pulse pressure of the subject by: determining, by analyzing the reflection of the ultrasound signal, a change in velocity of blood through the blood vessel between a systolic phase and a diastolic phase of the cardiac cycle; determining, by analyzing the reflection of the ultrasound signal, a pulse wave velocity (PWV) of the blood vessel; and determining the pulse pressure of the subject by: identifying an entry of a look-up table corresponding to the change in the velocity of the blood through the blood vessel and the PWV; or calculating the pulse pressure of the subject as a function of the change in the velocity of the blood through the blood vessel and the PWV.

11. The medical device of any of clauses 4 to 10, further including: an accelerometer physically coupled with the ultrasound transducer and configured to detect an acceleration of the medical device, wherein the processor is further configured to: determine that the acceleration of the medical device is below a threshold; and in response to determining that the acceleration of the medical device is below the threshold, cause the ultrasound transducer to output the ultrasound signal.

12. The medical device of any of clauses 4 to 11, further including: an output device configured to output an indication of the blood pressure.

13. A method, including: outputting, toward a blood vessel of a subject during a cardiac cycle of the subject, an ultrasound signal; detecting, from the blood vessel of the subject during the cardiac cycle of the subject, a reflection of the ultrasound signal; determining a normalized distension of the blood vessel of the subject during the cardiac cycle by analyzing the reflection of the ultrasound signal; determining a blood pressure of the subject by adding a mean arterial pressure of the subject to a product of the normalized distension of the blood vessel and a pulse pressure of the subject; and outputting an indication of the blood pressure.

14. The method of clause 13, wherein determining the normalized distension of the blood vessel of the subject during the cardiac cycle includes: determining, by analyzing the reflection of the ultrasound signal, a width metric of the blood vessel during the cardiac cycle, the width metric including a diameter of the blood vessel, a radius of the blood vessel, or a cross-sectional area of the blood vessel; determining a normalized width metric of the blood vessel during the cardiac cycle by dividing the width metric of the blood vessel during the cardiac cycle by a diastolic to systolic rise of the width metric of the blood vessel during the cardiac cycle; and in response to normalizing the width metric of the blood vessel during the cardiac cycle, determining the normalized distension of the blood vessel by subtracting the mean of the normalized width metric of the blood vessel during the cardiac cycle from the normalized width metric.

15. The method of clause 13 or 14, the blood vessel including an artery, wherein determining the mean arterial pressure of the subject includes: determining a mean of a velocity of blood through the artery by analyzing the reflection of the ultrasound signal; determining a diameter of the artery by analyzing the reflection of the ultrasound signal; and determining the mean arterial pressure of the subject by: identifying an entry of a look-up table or equation corresponding to the mean of the velocity of blood through the artery and the diameter of the artery; or calculating the mean arterial pressure of the subject as a function of the mean of the velocity of blood through the artery and the diameter of the artery.

16. The method of clause 15, further including: identifying a size of the subject, wherein the entry of the look-up table or equation further corresponds to the size of the subject or the function is further of the size of the subject.

17. The method of clause 16, wherein the size of the subject includes a BMI of the subject, a body fat percentage, or a clothing size of the subject.

18. The method of any of clauses 13 to 17, further including: determine the pulse pressure of the subject by: determining, by analyzing the reflection of the ultrasound signal, a change in velocity of blood through the blood vessel between a systolic phase and a diastolic phase of the cardiac cycle; determining, by analyzing the reflection of the ultrasound signal, a pulse wave velocity (PWV) of the blood vessel; and determining the pulse pressure of the subject by: identifying an entry of a look-up table corresponding to the change in the velocity of the blood through the blood vessel and the PWV; or calculating the pulse pressure of the subject as a function of the change in the velocity of the blood through the blood vessel and the PWV.

19. The method of any of clauses 13 to 18, further including: detecting an acceleration of a device that is configured to output the ultrasound signal; determining that the acceleration of the device is below a threshold; and in response to determining that the acceleration of the device is below the threshold, causing the device to output the ultrasound signal.

20. The method of any of clauses 13 to 19, the blood pressure being a first blood pressure estimation, the method further including: detecting, by a blood pressure cuff, a second blood pressure estimation of the subject; determining that the first blood pressure estimation has a greater accuracy than the second blood pressure estimation, wherein outputting the indication of the first blood pressure estimation is in response to determining that the first blood pressure estimation has the greater accuracy than the second blood pressure estimation.

21. A blood pressure monitor, including: an ultrasound transducer configured to: generate an ultrasound signal; and detect a reflection of the ultrasound signal from an artery of a subject during a cardiac cycle of the subject; and a processor configured to: determine a velocity of blood through the artery of the subject during the cardiac cycle by analyzing the reflection of the ultrasound signal; determine a pulse pressure of the subject by: determining a change in the velocity of blood through the artery of the subject between a systolic phase and a diastolic phase of the cardiac cycle; and determining a pulse wave velocity of the artery of the subject; and output the pulse pressure of the subject.

22. A blood pressure monitor, including: an ultrasound transducer configured to: generate an ultrasound signal; and detect a reflection of the ultrasound signal from an artery of a subject during a cardiac cycle of the subject; and a processor configured to: determine a velocity of blood through the artery of the subject during the cardiac cycle by analyzing the reflection of the ultrasound signal; determine a mean arterial pressure of the subject by: determining a mean of the velocity of the blood of through the artery of the subject during the cardiac cycle; and determining a mean of a distension waveform of the artery of the subject during the cardiac cycle; and output the mean arterial pressure of the subject.

23. A blood pressure monitor, including: an ultrasound transducer configured to: generate an ultrasound signal; and detect a reflection of the ultrasound signal from an artery of a subject during a cardiac cycle of the subject; an input device configured to receive an indication of a size of the subject; a display; and a processor configured to: determine a velocity of blood through the artery of the subject during the cardiac cycle by analyzing the reflection of the ultrasound signal; determine a pulse pressure of the subject by: determining a change in the velocity of blood through the artery of the subject between a systolic phase and a diastolic phase of the cardiac cycle; and calculating the pulse pressure as a function of the change in the velocity of blood through the artery of the subject and the size of the subject; determine a diameter of the artery of the subject during the cardiac cycle by analyzing the reflection of the ultrasound signal; determine a mean arterial pressure of the subject by: determining a mean of the velocity of the blood of through the artery of the subject during the cardiac cycle; determining a mean of the diameter of the artery of the subject during the cardiac cycle; and calculating the mean arterial pressure of the subject as a function of the mean of the velocity, the mean of the diameter, and the size of the subject; determine a normalized diameter of the artery during the cardiac cycle by subtracting, from the diameter of the artery during the cardiac cycle, the mean of the diameter of the artery during the cardiac cycle; determine a normalized distension of the artery during the cardiac cycle by determining a difference between a maximum of the normalized diameter and a minimum of the normalized diameter; and determine a blood pressure of the subject by adding, to a product of the normalized distension and the pulse pressure, the mean arterial pressure; and cause the display to visually present the blood pressure of the subject.

24. The blood pressure monitor of clause 23, wherein the size of the subject includes a height of the subject, a weight of the subject, a height-weight ratio of the subject, a body fat percentage, or a body mass index (BMI) of the subject.

25. The blood pressure monitor of clause 23 or 24, the blood pressure of the subject including a first estimated blood pressure of the subject, the blood pressure monitor further including: a blood pressure cuff configured to determine a second estimated blood pressure of the subject, wherein the processor is further configured to: determine that the first estimated blood pressure of the subject is more likely to be accurate than the second estimated blood pressure of the subject.

26. A medical device, including: an ultrasound transducer configured to: generate an ultrasound signal; and detect a reflection of the ultrasound signal from a blood vessel of a subject during a cardiac cycle of the subject; an input device configured to receive a signal indicating a size of the subject; and a processor configured to: determine a velocity of blood through the blood vessel of the subject by analyzing the reflection of the ultrasound signal; determine a blood pressure parameter as a function of the velocity of blood through the blood vessel and the size of the subject; and output an indication of the blood pressure parameter.

27. The medical device of clause 26, wherein the input device includes: a sensor configured to detect a height or a weight of the subject, the size of the subject including the height or the weight of the subject.

28. The medical device of clause 26 or 27, wherein the input device includes: a transceiver configured to receive, from an electronic medical record (EMR) device, the signal indicating the size of the subject.

29. The medical device of any of clauses 26 to 28, wherein the input device is configured to receive the signal from a user.

30. The medical device of any of clauses 26 to 29, wherein the processor is configured to determine the velocity of the blood through the blood vessel of the subject by: determining a Doppler shift between the ultrasound signal and the reflection of the ultrasound signal; and determining the velocity of the blood through the blood vessel as a function of the Doppler shift.

31. The medical device of any of clauses 26 to 30, wherein the velocity of the blood through the blood vessel of the subject includes a difference between a velocity of the blood through the blood vessel during a systolic phase of the cardiac cycle and a velocity of the blood through the blood vessel during a diastolic phase of the cardiac cycle, and wherein the blood pressure parameter includes a pulse pressure of the subject.

32. The medical device of clause 31, wherein the processor is configured to determine the blood pressure parameter as a function of the velocity of blood through the blood vessel, the size of the subject, and a pulse wave velocity of the blood vessel.

33. The medical device of any of clauses 26 to 32, wherein the velocity of the blood through the blood vessel of the subject includes a mean of the velocity of the blood through the blood vessel during the cardiac cycle, and wherein the blood pressure parameter includes a mean arterial pressure of the subject.

34. The medical device of clause 33, wherein the processor is configured to determine the blood pressure parameter as a function of the velocity of the blood through the blood vessel, the size of the subject, and a diameter of the blood vessel.

35. A method, including: outputting, toward a blood vessel of a subject during a cardiac cycle of the subject, an ultrasound signal; detecting, from the blood vessel of the subject during the cardiac cycle of the subject, a reflection of the ultrasound signal; identifying a size of the subject; determining a velocity of blood through the blood vessel of the subject by analyzing the reflection of the ultrasound signal; determining a blood pressure parameter as a function of the velocity of blood through the blood vessel and the size of the subject; and outputting an indication of the blood pressure parameter.

36. The method of clause 35, wherein identifying the size of the subject includes: detecting a height or a weight of the subject; receiving, from an EMR device, a signal indicating the size of the subject; or receiving, from a user, the signal indicating the size of the subject.

37. The method of clause 35 or 36, wherein determining the velocity of the blood through the blood vessel of the subject includes: determining a Doppler shift between the ultrasound signal and the reflection of the ultrasound signal; and determining the velocity of the blood through the blood vessel as a function of the Doppler shift.

38. The method of any of clauses 35 to 37, wherein the velocity of the blood through the blood vessel of the subject includes a difference between a velocity of the blood through the blood vessel during a systolic phase of the cardiac cycle and a velocity of the blood through the blood vessel during a diastolic phase of the cardiac cycle, and wherein the blood pressure parameter includes a pulse pressure of the subject.

39. The method of clause 38, wherein the blood pressure parameter includes a function of the velocity of blood through the blood vessel, the size of the subject, and a pulse wave velocity of the blood vessel.

40. The method of any of clauses 35 to 39, wherein the velocity of the blood through the blood vessel of the subject includes a mean of the velocity of the blood through the blood vessel during the cardiac cycle, and wherein the blood pressure parameter includes a mean arterial pressure of the subject.

41. The method of clause 40, wherein the blood pressure parameter is a function of the velocity of the blood through the blood vessel, the size of the subject, and a diameter of the blood vessel.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A blood pressure monitor, comprising:
an ultrasound transducer configured to:
generate an ultrasound signal; and
detect a reflection of the ultrasound signal from an artery of a subject during a cardiac cycle of the subject;
a display; and
a processor configured to:
determine a velocity of blood through the artery of the subject during the cardiac cycle by analyzing the reflection of the ultrasound signal;
determine a distension waveform of the artery of the subject during the cardiac cycle by analyzing the reflection of the ultrasound signal;
determine a pulse pressure of the subject by:
determining a change in the velocity of blood through the artery of the subject between a systolic phase and a diastolic phase of the cardiac cycle;
determining a pulse wave velocity of blood of the artery of the subject;
determine a mean arterial pressure of the subject by:
determining an average of the velocity of the blood through the artery of the subject during the cardiac cycle; and
determining an average of the distension waveform of the artery of the subject during the cardiac cycle;
determine a normalized distension waveform of the artery during the cardiac cycle by:
subtracting, from the distension waveform of the artery during the cardiac cycle, a minimum of the distension waveform of the artery during the cardiac cycle; and
in response to subtracting the minimum of the distension waveform of the artery, generating an intermediary waveform by dividing the distension waveform of the artery during the cardiac cycle by a maximum change in the distension waveform of the artery during the cardiac cycle; and
subtracting, from the intermediary waveform, the average of the distension waveform of the artery during the cardiac cycle;
and
determine a blood pressure waveform of the subject by adding, to a product of the normalized distension waveform and the pulse pressure, the mean arterial pressure; and
cause the display to visually present the blood pressure waveform of the subject.

2. The blood pressure monitor of claim 1, wherein the processor is configured to determine the pulse pressure of the subject by:
determining a vasculature load of the subject as a function of a size of the subject, a height of the subject, a weight of the subject, a height-weight ratio of the subject, a body fat percentage, or a body mass index (BMI) of the subject; and
determining the pulse pressure by:
identifying an entry of a look-up table corresponding to the change in the velocity of blood through the artery, the pulse wave velocity, and the vasculature load; or
calculating the pulse pressure as a function of the change in the velocity of blood through the artery, the pulse wave velocity, and the vasculature load.

3. The blood pressure monitor of claim 2, the look-up table being a first look-up table, wherein the processor is configured to determine the mean arterial pressure of the subject by:
identifying an entry of a second look-up table corresponding to the mean of the velocity through the artery during the cardiac cycle, the mean of the distension waveform of the artery during the cardiac cycle, and the vasculature load; or
calculating the mean arterial pressure of the subject as a function of the mean of the velocity through the artery during the cardiac cycle, the mean of the distension of the artery during the cardiac cycle, and the vasculature load.

4. A medical device, comprising:
an ultrasound transducer configured to:
output, toward a blood vessel of a subject during a cardiac cycle of the subject, an ultrasound signal; and
detect, from the blood vessel of the subject during the cardiac cycle of the subject, a reflection of the ultrasound signal; and
a processor configured to:
determine a normalized distension of the blood vessel of the subject during the cardiac cycle by analyzing the reflection of the ultrasound signal;
determine a blood pressure of the subject by adding a mean arterial pressure of the subject to a product of the normalized distension of the blood vessel and a pulse pressure of the subject; and
output an indication of the blood pressure.

5. The medical device of claim 4, wherein the processor is configured to determine the normalized distension of the blood vessel of the subject during the cardiac cycle by:
determining, by analyzing the reflection of the ultrasound signal, a width metric of the blood vessel during the cardiac cycle, the width metric comprising a diameter of the blood vessel, a radius of the blood vessel, or a cross-sectional area of the blood vessel;
determining a normalized width metric of the blood vessel during the cardiac cycle by dividing the width metric of the blood vessel during the cardiac cycle by a maximum change of the width metric of the blood vessel during the cardiac cycle; and
determining the normalized distension of the blood vessel by subtracting the mean of the width metric of the blood vessel during the cardiac cycle from the normalized width metric.

6. The medical device of claim 4, the blood vessel comprising an artery, wherein the processor is further configured to determine the mean arterial pressure of the subject by:
determining a mean of a velocity of blood through the artery by analyzing the reflection of the ultrasound signal;
determining a diameter of the artery by analyzing the reflection of the ultrasound signal; and
determining the mean arterial pressure of the subject by:
identifying an entry of a look-up table corresponding to the mean of the velocity of blood through the artery and the diameter of the artery; or
calculating the mean arterial pressure of the subject as a function of the mean of the velocity of blood through the artery and the diameter of the artery.

7. The medical device of claim 6, wherein determining the mean of the velocity of blood through the artery comprises:
determining a Doppler shift by identifying a difference between a frequency or phase of the ultrasound signal and a frequency or phase of the reflection of the ultrasound signal, the reflection of the ultrasound signal being from blood disposed in the artery; and
determining the velocity of blood through the artery as a function of the Doppler shift.

8. The medical device of claim 6, wherein the processor is further configured to:
identifying a size of the subject,
wherein the entry of the look-up table further corresponds to the size of the subject or the function is further dependent on the size of the subject.

9. The medical device of claim 8, wherein the size of the subject comprises a body mass index (BMI) of the subject or a clothing size of the subject.

10. The medical device of claim 4, wherein the processor is further configured to:
determine the pulse pressure of the subject by:
determining, by analyzing the reflection of the ultrasound signal, a change in velocity of blood through the blood vessel between a systolic phase and a diastolic phase of the cardiac cycle;
determining, by analyzing the reflection of the ultrasound signal, a pulse wave velocity (PWV) of the blood vessel; and
determining the pulse pressure of the subject by:
identifying an entry of a look-up table corresponding to the change in the velocity of the blood through the blood vessel and the PWV; or
calculating the pulse pressure of the subject as a function of the change in the velocity of the blood through the blood vessel and the PWV.

11. The medical device of claim 4, further comprising:
an accelerometer physically coupled with the ultrasound transducer and configured to detect an acceleration of the medical device,
wherein the processor is further configured to:
determine that the acceleration of the medical device is below a threshold; and
in response to determining that the acceleration of the medical device is below the threshold, cause the ultrasound transducer to output the ultrasound signal.

12. The medical device of claim 4, further comprising:
an output device configured to output an indication of the blood pressure.

13. A method, comprising:
outputting, toward a blood vessel of a subject during a cardiac cycle of the subject, an ultrasound signal;
detecting, from the blood vessel of the subject during the cardiac cycle of the subject, a reflection of the ultrasound signal;
determining a normalized distension of the blood vessel of the subject during the cardiac cycle by analyzing the reflection of the ultrasound signal;
determining a blood pressure of the subject by adding a mean arterial pressure of the subject to a product of the normalized distension of the blood vessel and a pulse pressure of the subject; and
outputting an indication of the blood pressure.

14. The method of claim 13, wherein determining the normalized distension of the blood vessel of the subject during the cardiac cycle comprises:
determining, by analyzing the reflection of the ultrasound signal, a width metric of the blood vessel during the cardiac cycle, the width metric comprising a diameter of the blood vessel, a radius of the blood vessel, or a cross-sectional area of the blood vessel;
determining a normalized width metric of the blood vessel during the cardiac cycle by dividing the width metric of the blood vessel during the cardiac cycle by a maximum change of the width metric of the blood vessel during the cardiac cycle; and
in response to normalizing the width metric of the blood vessel during the cardiac cycle, determining the normalized distension of the blood vessel by subtracting the mean of the width metric of the blood vessel during the cardiac cycle from the normalized width metric.

15. The method of claim 13, the blood vessel comprising an artery, wherein determining the mean arterial pressure of the subject comprises:
determining a mean of a velocity of blood through the artery by analyzing the reflection of the ultrasound signal;
determining a diameter of the artery by analyzing the reflection of the ultrasound signal; and
determining the mean arterial pressure of the subject by:
identifying an entry of a look-up table corresponding to the mean of the velocity of blood through the artery and the diameter of the artery; or
calculating the mean arterial pressure of the subject as a function of the mean of the velocity of blood through the artery and the diameter of the artery.

16. The method of claim 15, further comprising:
identifying a size of the subject,
wherein the entry of the look-up table further corresponds to the size of the subject or the function is further of the size of the subject.

17. The method of claim 16, wherein the size of the subject comprises a BMI of the subject or a clothing size of the subject.

18. The method of claim 13, further comprising:
determine the pulse pressure of the subject by:
determining, by analyzing the reflection of the ultrasound signal, a change in velocity of blood through the blood vessel between a systolic phase and a diastolic phase of the cardiac cycle;
determining, by analyzing the reflection of the ultrasound signal, a pulse wave velocity (PWV) of the blood vessel; and
determining the pulse pressure of the subject by:
identifying an entry of a look-up table corresponding to the change in the velocity of the blood through the blood vessel and the PWV; or
calculating the pulse pressure of the subject as a function of the change in the velocity of the blood through the blood vessel and the PWV.

19. The method of claim 13, further comprising:
detecting an acceleration of a device that is configured to output the ultrasound signal;
determining that the acceleration of the device is below a threshold; and
in response to determining that the acceleration of the device is below the threshold, causing the device to output the ultrasound signal.

20. The method of claim 13, the blood pressure being a first blood pressure estimation, the method further comprising:
detecting, by a blood pressure cuff, a second blood pressure estimation of the subject;
determining that the first blood pressure estimation has a greater accuracy than the second blood pressure estimation,
wherein outputting the indication of the first blood pressure estimation is in response to determining that the first blood pressure estimation has the greater accuracy than the second blood pressure estimation.
